(19) United States
(12) Patent Application Publication
(10) Pub. No.: US 2025/0127962 A1
(43) Pub. Date: Apr. 24, 2025

(54) GEL COMPRISING A HYDROGEL AND FAT OR A FAT DERIVATIVE IN THE FORM OF AN EMULSION

(71) Applicant: LABORATOIRES VIVACY, Paris (FR)

(72) Inventor: Stéphane SMARRITO, Vevey (CH)

(73) Assignee: LABORATOIRES VIVACY, Paris (FR)

(21) Appl. No.: 18/852,813

(22) PCT Filed: Mar. 30, 2023
(86) PCT No.: PCT/EP2023/058419

§ 371 Date: Nov. 25, 2024

(30) Foreign Application Priority Data
Mar. 30, 2022 (FR) FR2202907

(51) Int. Cl. A61L 27/52; A61L 27/20; A61L 27/24; A61L 27/26; A61L 27/48; A61L 27/54; C08L 5/08

(52) U.S. Cl. CPC A61L 27/52; A61L 27/20; A61L 27/24; A61L 27/26; A61L 27/48; A61L 27/54; C08L 5/08

(57) ABSTRACT

An emulsion, a gelled emulsion, or a gel include a hydrogel and fat or a fat derivative. The emulsion further includes at least one active ingredient selected from the group made of local anesthetics, vitamin C derivatives, anti-inflammatoires, antioxidants, and mixtures thereof. The method for preparing the emulsions, gelled emulsions, or gels includes the following steps: providing fat or fat derivative; providing a hydrogel; the hydrogel and fat or fat derivative are mixed aseptically; and the mixture is emulsified until an emulsion, gelled emulsion, or homogeneous gel is obtained. A kit for preparing an emulsion, a gelled emulsion, or a gel of fat or fat derivative and hyaluronic acid includes a cartridge and at least 2 syringes, making it possible to ensure the process, and the gel obtained, are sterile.

The present invention relates to a malleable gel comprising a hydrogel and fat or a fat derivative in emulsion form.

It thus concerns a gelled emulsion comprising a hydrogel and fat or a fat derivative.

It also relates to an emulsion comprising a hydrogel and fat or a fat derivative.

Fat or fat derivative is understood to mean fat for lipofilling, MICROFAT, or NANOFAT.

In one embodiment, the fat is fat for lipofilling, MICROFAT, or NANOFAT.

Fat for lipofilling is fat consisting of fatty tissue which is removed using a cannula and has not undergone any transformation or alteration.

MICROFAT is fat consisting of fatty tissue which is removed using a small-diameter cannula and has not undergone any transformation or alteration.

NANOFAT is fat made up of removed fatty tissue which has undergone mechanical action with knives in order to liquefy it, followed by filtration, to obtain a highly fluid, cell-enriched substance.

NANOFAT can also be obtained using kits designed to treat adipose tissue for the purpose of releasing the stromal vascular fraction from adipose tissue cells.

In one embodiment, the fat is fat for lipofilling.

In one embodiment, the fat is MICROFAT.

In one embodiment, the fat is NANOFAT.

The emulsion obtained is in the form of a composition as illustrated in Example 7 whose viscoelastic properties are those of a gel whose G′ is greater than or equal to 20 Pa.

In one embodiment, G′ is greater than or equal to 30 Pa.

In one embodiment, G′ is greater than or equal to 40 Pa.

The emulsion according to the invention is therefore a gelled emulsion or a gel.

In one embodiment, the gel, gelled emulsion, or the emulsion according to the invention is characterized in that the volume ratio of hydrogel/fat or fat derivative is between 0.5/4.5 and 2/3.

In one embodiment, the gel, gelled emulsion, or the emulsion according to the invention is characterized in that the volume ratio of hydrogel/fat or fat derivative is 1/4.

The hydrogel consists of at least one polysaccharide selected from the group consisting of hyaluronic acid, keratan, heparin, cellulose, cellulose derivatives (particularly hydroxypropyl cellulose, hydroxypropylmethyl cellulose, ethylmethyl cellulose, carboxymethyl cellulose), alginic acid, xanthan, carrageenan, chitosan, chondroitin, heparosan, and biologically acceptable salts thereof, alone or as a mixture.

In one embodiment, said polysaccharide is hyaluronic acid.

In one embodiment, said polysaccharide is hyaluronic acid or a salt thereof, alone or as a mixture.

In one embodiment, said polysaccharide is hyaluronic acid in the form of sodium or potassium salt.

In one embodiment, said polysaccharide is hyaluronic acid in the form of sodium salt.

In one embodiment, said polysaccharide is non-crosslinked hyaluronic acid or a salt thereof, alone or as a mixture.

In one embodiment, said polysaccharide is crosslinked hyaluronic acid or a salt thereof, alone or as a mixture.

In one embodiment, said polysaccharide is crosslinked hyaluronic acid or a salt thereof, alone or as a mixture, said crosslinking being carried out by means of at least one crosslinking agent.

In one embodiment, said polysaccharide is crosslinked hyaluronic acid or a salt thereof, alone or as a mixture, said crosslinking being carried out by means of at least one bi- or polyfunctional crosslinking agent.

In one embodiment, said polysaccharide is crosslinked hyaluronic acid or a salt thereof, alone or as a mixture, said crosslinking being carried out by means of at least one bifunctional crosslinking agent, either butanediol diglycidyl ether (BDDE) or 1,2,7,8-diepoxyoctane.

In one embodiment, said polysaccharide is crosslinked hyaluronic acid or a salt thereof, alone or as a mixture, said crosslinking being carried out by means of BDDE.

In one embodiment, said polysaccharide is crosslinked hyaluronic acid or a salt thereof, alone or as a mixture, said crosslinking being carried out by means of at least sodium or potassium trimetaphosphate.

When the polysaccharide and, more particularly, hyaluronic acid is crosslinked using a crosslinking agent, the crosslinking rate (x) is calculated theoretically using the following formula:

X=number of moles of crosslinking agent introduced into the reaction medium/number of moles of repeat units introduced into the reaction medium

In hyaluronic acid, the repeating unit is a disaccharide motif.

In one embodiment, the crosslinked hyaluronic acid has a crosslinking rate x of between 0.001 and 0.5.

In one embodiment, the crosslinked hyaluronic acid has a crosslinking rate x of between 0.01 and 0.4.

In one embodiment, the crosslinked hyaluronic acid has a crosslinking rate x of between 0.1 and 0.3.

In one embodiment, the crosslinked hyaluronic acid has a crosslinking ratio x of 0.06.

In one embodiment, the crosslinked hyaluronic acid has a crosslinking ratio x of 0.07.

In one embodiment, the crosslinked hyaluronic acid has a crosslinking ratio x of 0.12.

In one embodiment, said polysaccharide is co-crosslinked hyaluronic acid or a salt thereof, alone or as a mixture.

In one embodiment, said polysaccharide is crosslinked or non-crosslinked hyaluronic acid that has been chemically modified by substitution, or a salt thereof, alone or as a mixture.

In one embodiment, said polysaccharide is doubly crosslinked hyaluronic acid as described in patent application WO2000/046253 in the name of Fermentech Medical Limited.

In one embodiment, said polysaccharide is a mixture of crosslinked and non-crosslinked hyaluronic acids, or a salt thereof.

In one embodiment, said polysaccharide is a mixture of crosslinked hyaluronic acids, or a salt thereof.

In one embodiment, said polysaccharide is a mixture of crosslinked hyaluronic acids, or a salt thereof, such as that described in patent application WO2009/071697 in the applicant's name.

In one embodiment, said polysaccharide is a mixture of hyaluronic acids obtained by mixing a plurality of hyaluronic acids, or a salt thereof, of different molecular weights prior to crosslinking, as described in patent application WO2004092222 in the name of Cornéal Industrie.

In one embodiment, said polysaccharide is hyaluronic acid, or a salt thereof, substituted by a group which imparts lipophilic or moisturizing properties, such as, for example, substituted hyaluronic acids as described in patent application FR2983483 in the applicant's name. That application describes a method for the simultaneous substitution and crosslinking of a polysaccharide via its hydroxyl functions, in the aqueous phase, characterized in that it comprises the following steps: (i) a polysaccharide is placed in an aqueous medium, (ii) it is placed in the presence of at least one precursor of a substituent, (iii) it is placed in the presence of a crosslinking agent, and (iv) the substituted and crosslinked polysaccharide is obtained and isolated. These method steps can be perfectly implemented within the framework of the present invention.

In one embodiment, said polysaccharide is hyaluronic acid or a salt thereof, grafted with glycerol, for example as described in application WO2017162676 in the name of MERZ.

In one embodiment, the molecular weight Mw of the at least one hyaluronic acid is within a range of from 0.01 MDa to 5 MDa (0.01≤Mw≤5 MDa).

In one embodiment, the molecular weight Mw of the at least one hyaluronic acid is within a range of from 0.1 MDa to 3.5 MDa (0.1≤Mw≤3.5 MDa).

In one embodiment, the average molecular weight Mw of the at least one hyaluronic acid is within a range of from 1 MDa to 3 MDa (1≤Mw≤3 MDa).

In one embodiment, the molecular weight Mw of the at least one hyaluronic acid is 1 MDa.

In one embodiment, the molecular weight Mw of the at least one hyaluronic acid is 3 MDa.

In one embodiment, the concentration of hyaluronic acid [HA] is between 2 mg/g and 50 mg/g of the total weight of said hydrogel.

In one embodiment, the concentration of hyaluronic acid [HA] is between 4 mg/g and 40 mg/g of the total weight of said hydrogel.

In one embodiment, the concentration of hyaluronic acid [HA] is between 5 mg/g and 30 mg/g of the total weight of said hydrogel.

In one embodiment, the concentration of hyaluronic acid [HA] is between 10 mg/g and 30 mg/g of the total weight of said hydrogel.

In one embodiment, the concentration of hyaluronic acid [HA] is 20 mg/g of the total weight of said hydrogel.

In one embodiment, the concentration of hyaluronic acid [HA] is 14 mg/g of the total weight of said hydrogel.

In one embodiment, the concentration of hyaluronic acid [HA] is 10 mg/g of the total weight of said hydrogel.

In one embodiment, the gel, gelled emulsion, or emulsion further comprises at least one active ingredient, the active ingredient being selected from the group consisting of local anesthetics, vitamin C derivatives, anti-inflammatories, antioxidants, and mixtures thereof.

In one embodiment the gel, gelled emulsion, or emulsion is free of emulsifiers, for example as described in patent application WO2011019822 in the name of JOHN HOPKINS.

In one embodiment, the gel, gelled emulsion, or emulsion is free of chemical compounds promoting vascularization (angiogenesis or vasculogenesis) as described in patent application US20120189708 in the name of ALLERGAN.

In one embodiment, the gel, gelled emulsion, or emulsion is free of polymers promoting cell viability or proliferation, such as silk fibers or collagen as described in application US20180055971 in the name of ALLERGAN, for example.

In one embodiment, the hydrogel is sterilized, meaning that it undergoes a sterilization step after its preparation, said sterilization step being carried out by heat, by moist heat, by gamma radiation (γ), by accelerated electron beam, or by sterilizing filtration (0.22 μm) in the case of hydrogels based on non-crosslinked hyaluronic acid.

In one embodiment, said sterilization step is carried out by steam autoclaving.

In one embodiment, the steam autoclaving is carried out at a temperature of from 121 to 134° C., for a duration appropriate to the temperature.

In one embodiment, the steam autoclaving is carried out at a temperature of between 127 and 130° C. for a duration of between 1 and 20 min.

In one embodiment, the sterilizing value F0 is greater than 1 min at 121° C.

In one embodiment, said sterilization step is carried out by irradiation by gamma (γ) rays.

In one embodiment, the method for preparing gels, gelled emulsions, or emulsions according to the invention further comprises at least one step of adding at least one active ingredient.

In one embodiment, the at least one active ingredient is added in powder form.

In one embodiment, the at least one active is added in the form of a solution or suspension.

In one embodiment, the at least one active ingredient is added in the form of a solution or suspension to a solvent or a solution selected from the group consisting of water, aqueous saline solutions, for example a phosphate buffer solution, for example PBS.

In one embodiment, the at least one active ingredient is added to hyaluronic acid.

In one embodiment, the at least one active ingredient is added afterward during the step of preparing the gels, gelled emulsions, or emulsions according to the invention.

In one embodiment, the method for preparing an emulsion, a gelled emulsion, or a gel according to the invention further comprises at least one step of adding at least one active ingredient selected from the group consisting of local anesthetics, vitamin C derivatives, anti-inflammatoires, antioxidants, and mixtures thereof.

In one embodiment, the method for preparing an emulsion, a gelled emulsion, or a gel according to the invention further comprises at least one step of adding at least one local anesthetic.

In one embodiment, the method for preparing an emulsion, a gelled emulsion, or a gel according to the invention further comprises at least one step of adding at least one local anesthetic to obtain a local anesthetic concentration of between 0.1 and 5% relative to the total mass of said emulsion.

In one embodiment, the method for preparing an emulsion, a gelled emulsion, or a gel according to the invention further comprises at least one step of adding at least one local anesthetic to obtain a local anesthetic concentration of between 0.1 and 4% relative to the total mass of said emulsion.

In one embodiment, the method for preparing an emulsion, a gelled emulsion, or a gel according to the invention further comprises at least one step of adding at least one local anesthetic to obtain a local anesthetic concentration of between 0.1 and 2% relative to the total mass of said emulsion.

In one embodiment, the method for preparing an emulsion, a gelled emulsion, or a gel according to the invention further comprises at least one step of adding at least one local anesthetic to obtain a local anesthetic concentration of between 0.1 and 1% relative to the total mass of said emulsion.

In one embodiment, the method for preparing an emulsion, a gelled emulsion, or a gel according to the invention further comprises at least one step of adding at least one local anesthetic to obtain a local anesthetic concentration of between 0.1 and 0.5% relative to the total mass of said emulsion.

In one embodiment, the method for preparing an emulsion, a gelled emulsion, or a gel according to the invention further comprises at least one step of adding at least one local anesthetic to obtain a concentration of local anesthetic of approximately 0.3% relative to the total mass of said emulsion.

In one embodiment, the local anesthetic is selected from the group of the amino esters.

In one embodiment, the amino ester is selected from the group consisting of procaine, benzocaine, chloroprocaine, and tetracaine in the form of a base or salt, for example in the form of a hydrochloride.

In one embodiment, the local anesthetic is selected from the group of the amino amides.

In one embodiment, the amino amide is selected from the group consisting of lidocaine, mepivacaine, prilocaine, articaine, aptocaine, bupivacaine, etidocaine, and ropivacaine in the form of a base or salt, for example in the form of a hydrochloride.

In one embodiment, the local anesthetic is selected from the group of the amino ethers.

In one embodiment, the amino ether is selected from the group consisting of diamocaine and pramocaine in the form of a base or salt, for example in the form of a hydrochloride or cyclamate.

In one embodiment, the amino ether is selected from the group consisting of lidocaine, mepivacaine, and salts and isolated isomers thereof.

In one embodiment, the amino ether is lidocaine.

In one embodiment, the amino ether is lidocaine or a pharmaceutically acceptable salt thereof.

In one embodiment, the amino ether is lidocaine hydrochloride.

In one embodiment, the amino ether is mepivacaine.

In one embodiment, the amino ether is mepivacaine or a pharmaceutically acceptable salt thereof.

In one embodiment, the amino ether is selected from the group consisting of racemic mepivacaine hydrochloride, (r)-mepivacaine hydrochloride, (s)-mepivacaine hydrochloride, (r)-mepivacaine, and(s)-mepivacaine, or a pharmaceutically acceptable salt thereof.

In one embodiment, the amino ether is mepivacaine hydrochloride.

In one embodiment, the amino ether is (r)-mepivacaine hydrochloride.

In one embodiment, the amino ether is(s)-mepivacaine hydrochloride.

In one embodiment, the amino ether is racemic mepivacaine hydrochloride.

In one embodiment, the amino ether is (r)-mepivacaine.

In one embodiment, the amino ether is(s)-mepivacaine.

In one embodiment, the amino ether is racemic mepivacaine.

In one embodiment, the method for preparing an emulsion, a gelled emulsion, or a gel according to the invention further comprises at least one step of adding at least one amino ether local anesthetic selected from the group consisting of lidocaine, mepivacaine, and mixtures thereof.

In one embodiment, the method for preparing an emulsion, a gelled emulsion, or a gel according to the invention further comprises at least one step of adding at least one local anesthetic, namely lidocaine.

In one embodiment, the method for preparing an emulsion, a gelled emulsion, or a gel according to the invention further comprises at least one step of adding at least one local anesthetic, namely lidocaine, to obtain a lidocaine concentration of between 0.1 and 5% relative to the total mass of said emulsion.

In one embodiment, the method for preparing an emulsion, a gelled emulsion, or a gel according to the invention further comprises at least one step of adding at least one local anesthetic, namely lidocaine, to obtain a lidocaine concentration of between 0.1 and 4% relative to the total mass of said emulsion.

In one embodiment, the method for preparing an emulsion, a gelled emulsion, or a gel according to the invention further comprises at least one step of adding at least one local anesthetic, namely lidocaine, to obtain a lidocaine concentration of between 0.1 and 2% relative to the total mass of said emulsion.

In one embodiment, the method for preparing an emulsion, a gelled emulsion, or a gel according to the invention further comprises at least one step of adding at least one local anesthetic, namely lidocaine, to obtain a lidocaine concentration of between 0.1 and 1% relative to the total mass of said emulsion.

In one embodiment, the method for preparing an emulsion, a gelled emulsion, or a gel according to the invention further comprises at least one step of adding at least one local anesthetic, namely lidocaine, to obtain a lidocaine concentration of between 0.1 and 0.5% relative to the total mass of said emulsion.

In one embodiment, the method for preparing an emulsion, a gelled emulsion, or a gel according to the invention further comprises at least one step of adding at least one local anesthetic, namely lidocaine, to obtain a local anesthetic concentration of approximately 0.3% relative to the total mass of said emulsion.

In one embodiment, the method for preparing an emulsion, a gelled emulsion, or a gel according to the invention further comprises at least one step of adding at least one local anesthetic, namely mepivacaine.

In one embodiment, the method for preparing an emulsion, a gelled emulsion, or a gel according to the invention further comprises at least one step of adding at least one local anesthetic, namely mepivacaine, to obtain a mepivacaine concentration of between 0.1 and 5% relative to the total mass of said emulsion.

In one embodiment, the method for preparing an emulsion, a gelled emulsion, or a gel according to the invention further comprises at least one step of adding at least one local anesthetic, namely mepivacaine, to obtain a mepivacaine concentration of between 0.1 and 4% relative to the total mass of said emulsion.

In one embodiment, the method for preparing an emulsion, a gelled emulsion, or a gel according to the invention further comprises at least one step of adding at least one local anesthetic, namely mepivacaine, to obtain a mepivacaine concentration of between 0.1 and 2% relative to the total mass of said emulsion.

In one embodiment, the method for preparing an emulsion, a gelled emulsion, or a gel according to the invention further comprises at least one step of adding at least one local anesthetic, namely mepivacaine, to obtain a mepivacaine concentration of between 0.1 and 1% relative to the total mass of said emulsion.

In one embodiment, the method for preparing an emulsion, a gelled emulsion, or a gel according to the invention further comprises at least one step of adding at least one local anesthetic, namely mepivacaine, to obtain a mepivacaine concentration of between 0.1 and 0.5% relative to the total mass of said emulsion.

In one embodiment, the method for preparing an emulsion, a gelled emulsion, or a gel according to the invention further comprises at least one step of adding at least one local anesthetic, namely mepivacaine, to obtain a local anesthetic concentration of approximately 0.3% relative to the total mass of said gel, gelled emulsion, or emulsion.

In one embodiment, the method for preparing an emulsion, a gelled emulsion, or a gel according to the invention further comprises at least one step of adding at least one local anesthetic, namely dyclonine, in the form of a base or salt, for example in the form of a hydrochloride.

In one embodiment, the method for preparing an emulsion, a gelled emulsion, or a gel according to the invention further comprises at least one step of adding at least one local anesthetic selected from the group consisting of chlorobutanol, guafecainol, and polidocanol.

In one embodiment, the local anaesthetic is chlorobutanol.

In one embodiment, the local anaesthetic is guafecainol.

In one embodiment, the local anaesthetic is polidocanol.

In one embodiment, the method for preparing an emulsion, a gelled emulsion, or a gel according to the invention further comprises at least one step of adding at least one anti-inflammatory.

In one embodiment, the method for preparing an emulsion, a gelled emulsion, or a gel according to the invention further comprises at least one step of adding at least one anti-inflammatory selected from the group consisting of the steroidal and nonsteroidal anti-inflammatory drugs.

In one embodiment, the method for preparing an emulsion, a gelled emulsion, or a gel according to the invention further comprises at least one step of adding at least one anti-inflammatory selected from the group consisting of the nonsteroidal anti-inflammatory drugs.

In one embodiment, the method for preparing an emulsion, a gelled emulsion, or a gel according to the invention further comprises at least one step of adding at least one anti-inflammatory selected from the group comprising salicylated anti-inflammatoires, propionic derivatives, indole derivatives, pyrazole derivatives, oxicams, and coxibs.

In one embodiment, the method for preparing an emulsion, a gelled emulsion, or a gel according to the invention further comprises at least one step of adding at least one anti-inflammatory selected from the group comprising diclofenac, nimesufide, niflumic acid, mefenamic acid, and nabumetone, alone or as a mixture.

In one embodiment, the method for preparing an emulsion, a gelled emulsion, or a gel according to the invention further comprises at least one step of adding at least one salicylated anti-inflammatory selected from the group comprising diflunisal, benorilate, and aspirin, alone or as a mixture.

In one embodiment, the method for preparing an emulsion, a gelled emulsion, or a gel according to the invention further comprises at least one step of adding at least one anti-inflammatory selected from the group of the propionic derivatives comprising alminoprofen, ketoprofen, ibuprofen, naproxen, flurbiprofen and tiaprofenic acid, alone or as a mixture.

In one embodiment, the method for preparing an emulsion, a gelled emulsion, or a gel according to the invention further comprises at least one step of adding at least one anti-inflammatory selected from the group of the indole derivatives comprising indomethacin, sulindac, and etodolac, alone or as a mixture.

In one embodiment, the method for preparing an emulsion, a gelled emulsion, or a gel according to the invention further comprises at least one step of adding at least one anti-inflammatory selected from the group of the pyrazole derivatives comprising, in particular, phenylbutazone.

In one embodiment, the method for preparing an emulsion, a gelled emulsion, or a gel according to the invention further comprises at least one step of adding at least one anti-inflammatory selected from the group of the oxicams comprising piroxicam, tenoxicam, and meloxicam, alone or as a mixture.

In one embodiment, the method for preparing an emulsion, a gelled emulsion, or a gel according to the invention further comprises at least one step of adding at least one anti-inflammatory selected from the group of the coxibs comprising celecoxib, etoricoxib, and rofecoxib, alone or as a mixture.

In one embodiment, the concentration of nonsteroidal anti-inflammatory drugs in the gel, gelled emulsion, or emulsion according to the invention is between 0.01 and 2000 mg/g.

In one embodiment, the concentration of nonsteroidal anti-inflammatory drugs in the gel, gelled emulsion, or emulsion according to the invention is between 0.1 and 1000 mg/g.

In one embodiment, the concentration of nonsteroidal anti-inflammatory drugs in the gel, gelled emulsion, or emulsion according to the invention is between 0.5 and 500 mg/g.

In one embodiment, the method for preparing an emulsion, a gelled emulsion, or a gel according to the invention further comprises at least one step of adding at least one steroidal anti-inflammatory drug.

In one embodiment, the method for preparing an emulsion, a gelled emulsion, or a gel according to the invention further comprises at least one step of adding at least one steroidal anti-inflammatory drug selected from the group comprising dexamethasone, prednisolone, corticosterone, budesonide, sulfasalazine, mesalamine, cetirizine, diphenhydramine, antipyrine, methyl salicylate, loratadine, thymol, carvacrol, bisabolol, allantoin, eucalyptol, phenazone (antipyrine), propyphenazone, alone or as a mixture.

In one embodiment, the concentration of steroidal anti-inflammatoires in the gel, gelled emulsion, or emulsion according to the invention is between 0.01 and 2000 mg/g.

In one embodiment, the concentration of steroidal anti-inflammatoires in the gel, gelled emulsion, or emulsion according to the invention is between 0.1 and 1000 mg/g.

In one embodiment, the concentration of steroidal anti-inflammatoires in the gel, gelled emulsion, or emulsion according to the invention is between 0.5 and 500 mg/g.

In one embodiment, the method for preparing an emulsion, a gelled emulsion, or a gel according to the invention further comprises at least one step of adding at least one anti-inflammatory selected from the group consisting of sucrose octasulfate and salts thereof.

In one embodiment, the method for preparing an emulsion, a gelled emulsion, or a gel according to the invention further comprises at least one step of adding at least one anti-inflammatory selected from the group consisting of sucrose octasulfate and sodium and potassium salts thereof.

In one embodiment, the method for preparing an emulsion, a gelled emulsion, or a gel according to the invention further comprises at least one step of adding at least one anti-inflammatory water-soluble salt of sucrose octasulfate selected from the group consisting of alkali metal salts, alkaline earth metal salts, silver salts, ammonium salts, amino acid salts.

In one embodiment, the method for preparing an emulsion, a gelled emulsion, or a gel according to the invention further comprises at least one step of adding at least one anti-inflammatory water-soluble salt of sucrose octasulfate selected from the group consisting of alkali metal salts or alkaline earth metal salts.

In one embodiment, the method for preparing an emulsion, a gelled emulsion, or a gel according to the invention further comprises at least one step of adding at least one anti-inflammatory water-soluble salt of sucrose octasulfate selected from the group consisting of the sodium salt of sucrose octasulfate or the potassium salt of sucrose octasulfate.

In one embodiment, the method for preparing an emulsion, a gelled emulsion, or a gel according to the invention further comprises at least one step of adding at least one antimicrobial.

In one embodiment, the method for preparing an emulsion, a gelled emulsion, or a gel according to the invention further comprises at least one step of adding at least one antimicrobial selected from the group comprising gentamicin, silver sulfadiazine, metronidazole, fucidin, bacitracin, eosin, povidone iodine, copper gluconate, zinc gluconate, manganese gluconate, or salts thereof, alone or as a mixture.

In one embodiment, the concentration of antimicrobials in the gel, gelled emulsion, or emulsion according to the invention is between 0.1 and 200 mg/g.

In one embodiment, the concentration of antimicrobials in the gel, gelled emulsion, or emulsion according to the invention is between 0.5 and 100 mg/g.

In one embodiment, the method for preparing an emulsion, a gelled emulsion, or a gel according to the invention further comprises at least one step of adding at least one glycoside or glycoside derivative.

In one embodiment, the method for preparing an emulsion, a gelled emulsion, or a gel according to the invention further comprises at least one step of adding at least one glycoside or a glycoside derivative selected from the group comprising D-glucopyranose, 1,4 glycoside, esculin, hesperidin, diosmin, arbutin, skimmin, or aloin, alone or in mixtures.

In one embodiment, the concentration of glycosides in the gel, gelled emulsion, or emulsion according to the invention is between 0.1 and 200 mg/g.

In one embodiment, the concentration of glycosides in the gel, gelled emulsion, or emulsion according to the invention is between 0.5 and 100 mg/g.

In one embodiment, the method for preparing an emulsion, a gelled emulsion, or a gel according to the invention further comprises at least one step of adding at least one moisturizer or tissue regenerator.

In one embodiment, the method for preparing an emulsion, a gelled emulsion, or a gel according to the invention further comprises at least one step of adding at least one moisturizer or tissue regenerator selected from the group consisting of the macroelements.

In one embodiment, the method for preparing an emulsion, a gelled emulsion, or a gel according to the invention further comprises at least one step of adding at least one macroelement selected from the group comprising iron, calcium, copper, zinc, manganese, magnesium or potassium gluconates, trimethylsilanol, trimethylsilanolate, potassium trimethylsilanolate, methylsilanol mannuronate, sodium monoethyl trisilanol ortho-hydroxybenzoate, alone or as a mixture.

In one embodiment, the method for preparing an emulsion, a gelled emulsion, or a gel according to the invention further comprises at least one step of adding at least one moisturizer or tissue regenerator selected from the group consisting of the essential amino acids.

In one embodiment, the method for preparing an emulsion, a gelled emulsion, or a gel according to the invention further comprises at least one step of adding at least one essential amino acid selected from the group comprising isoleucine, leucine, lysine, methionine, phenylalanine, threonine, tryptophan, and valine, alone or as a mixture.

In one embodiment, the method for preparing an emulsion, a gelled emulsion, or a gel according to the invention further comprises at least one step of adding at least one moisturizer or tissue regenerator selected from the group consisting of the semi-essential amino acids.

In one embodiment, the method for preparing an emulsion, a gelled emulsion, or a gel according to the invention further comprises at least one step of adding at least one semi-essential amino acid selected from the group comprising arginine and histidine, alone or as a mixture.

In one embodiment, the method for preparing an emulsion, a gelled emulsion, or a gel according to the invention further comprises at least one step of adding at least one moisturizer or tissue regenerator selected from the group consisting of the non-essential amino acids.

In one embodiment, the method for preparing an emulsion, a gelled emulsion, or a gel according to the invention further comprises at least one step of adding at least one non-essential amino acid selected from the group comprising alanine, asparagine, aspartic acid, cysteine, glutamine, glutamic acid, glycine, proline, serine, tyrosine, alone or as a mixture.

In one embodiment, the method for preparing an emulsion, a gelled emulsion, or a gel according to the invention further comprises at least one step of adding at least one moisturizer or tissue regenerator selected from the group consisting of the vitamins.

In one embodiment, the method for preparing an emulsion, a gelled emulsion, or a gel according to the invention further comprises at least one step of adding at least one vitamin selected from the group consisting of retinol, thiamine, riboflavin, nicotinamide, adenine, calcium pantothenate, pyridoxine, inositol, biotin, folic acid, para-aminobenzoic acid, cobalamin, vitamin C, choline chloride, alone or as a mixture.

In one embodiment, the method for preparing an emulsion, a gelled emulsion, or a gel according to the invention further comprises at least one step of adding at least one tissue moisturizer or regenerator selected from the group consisting of the nucleic acids.

In one embodiment, the method for preparing an emulsion, a gelled emulsion, or a gel according to the invention further comprises at least one step of adding at least one nucleic acid selected from the group consisting of deoxyadenosine, deoxycytidine, deoxyguanosine, deoxythymidine, methylcytosine, alone or as a mixture.

In one embodiment, the method for preparing an emulsion, a gelled emulsion, or a gel according to the invention further comprises at least one step of adding at least one moisturizer or tissue regenerator selected from the group consisting of the coenzymes.

In one embodiment, the method for preparing an emulsion, a gelled emulsion, or a gel according to the invention further comprises at least one step of adding at least one coenzyme selected from the group consisting of thiamine pyrophosphate, coenzyme A, FAD, NAD, NADP, UTP, alone or as a mixture.

In one embodiment, the method for preparing an emulsion, a gelled emulsion, or a gel according to the invention further comprises at least one step of adding at least one moisturizer or tissue regenerator selected from the group consisting of deoxythymidine, glutathione, sodium pyruvate, lipoic acid, and putrescine, alone or as a mixture.

In one embodiment, the concentration of tissue moisturizers or regenerators in the gel, gelled emulsion, or emulsion according to the invention is between 0.01 and 500 mg/g.

In one embodiment, the concentration of tissue moisturizers or regenerators in the gel, gelled emulsion, or emulsion according to the invention is between 0.1 and 200 mg/g.

In one embodiment, the method for preparing an emulsion, a gelled emulsion, or a gel according to the invention further comprises at least one step of adding at least one antioxidant.

In one embodiment, the method for preparing an emulsion, a gelled emulsion, or a gel according to the invention further comprises at least one step of adding at least one antioxidant selected from the group of the polyols.

In one embodiment, the method for preparing an emulsion, a gelled emulsion, or a gel according to the invention further comprises at least one step of adding at least one polyol selected from the group consisting of mannitol, sorbitol, propylene glycol, xylitol, glycerol, maltitol, lactitol, and erythritol.

In one embodiment, the method for preparing an emulsion, a gelled emulsion, or a gel according to the invention further comprises at least one step of adding at least one polyol selected from the group consisting of mannitol, sorbitol, maltitol, and glycerol, alone or as a mixture.

In one embodiment, the method for preparing an emulsion, a gelled emulsion, or a gel according to the invention further comprises at least one step of adding at least one polyol selected from the group consisting of mannitol, sorbitol, and maltitol, and salts thereof.

In one embodiment, the method for preparing an emulsion, a gelled emulsion, or a gel according to the invention further comprises at least one step of adding at least one polyol to obtain a polyol concentration of between 0.1 mg/ml and 50 mg/ml relative to the total mass of said emulsion.

In one embodiment, the method for preparing an emulsion, a gelled emulsion, or a gel according to the invention further comprises at least one step of adding at least one polyol to obtain a polyol concentration of between 5 mg/ml and 40 mg/ml relative to the total mass of said emulsion.

In one embodiment, the method for preparing an emulsion, a gelled emulsion, or a gel according to the invention further comprises at least one step of adding at least one polyol to obtain a polyol concentration of between 10 mg/ml and 40 mg/ml relative to the total mass of said emulsion.

In one embodiment, the method for preparing an emulsion, a gelled emulsion, or a gel according to the invention further comprises at least one step of adding at least one polyol to obtain a polyol concentration of between 20 mg/ml and 40 mg/ml relative to the total mass of said emulsion.

In one embodiment, the method for preparing an emulsion, a gelled emulsion, or a gel according to the invention further comprises at least one step of adding at least one polyol to obtain a polyol concentration of between 30 mg/ml and 40 mg/ml relative to the total mass of said emulsion.

In one embodiment, the method for preparing an emulsion, a gelled emulsion, or a gel according to the invention further comprises at least one step of adding at least one polyol, namely mannitol.

In one embodiment, the method for preparing an emulsion, a gelled emulsion, or a gel according to the invention further comprises at least one step of adding mannitol to obtain a mannitol concentration of between 5 mg/ml and 40 mg/ml relative to the total mass of said emulsion.

In one embodiment, the method for preparing an emulsion, a gelled emulsion, or a gel according to the invention further comprises at least one step of adding mannitol to obtain a mannitol concentration of between 10 mg/ml and 40 mg/ml relative to the total mass of said emulsion.

In one embodiment, the method for preparing an emulsion, a gelled emulsion, or a gel according to the invention further comprises at least one step of adding mannitol to obtain a mannitol concentration of between 20 mg/ml and 40 mg/ml relative to the total mass of said emulsion.

In one embodiment, the method for preparing an emulsion, a gelled emulsion, or a gel according to the invention further comprises at least one step of adding mannitol to obtain a mannitol concentration of between 30 mg/ml and 40 mg/ml relative to the total mass of said emulsion.

In one embodiment, the method for preparing an emulsion, a gelled emulsion, or a gel according to the invention further comprises at least one step of adding at least one polyol, namely sorbitol.

In one embodiment, the method for preparing an emulsion, a gelled emulsion, or a gel according to the invention further comprises at least one step of adding sorbitol to obtain a sorbitol concentration of between 5 mg/ml and 40 mg/ml relative to the total mass of said emulsion.

In one embodiment, the method for preparing an emulsion, a gelled emulsion, or a gel according to the invention further comprises at least one step of adding sorbitol to obtain a sorbitol concentration of between 10 mg/ml and 40 mg/ml relative to the total mass of said emulsion.

In one embodiment, the method for preparing an emulsion, a gelled emulsion, or a gel according to the invention further comprises at least one step of adding sorbitol to obtain a sorbitol concentration of between 20 mg/ml and 40 mg/ml relative to the total mass of said emulsion.

In one embodiment, the method for preparing an emulsion, a gelled emulsion, or a gel according to the invention further comprises at least one step of adding sorbitol to obtain a sorbitol concentration of between 30 mg/ml and 40 mg/ml relative to the total mass of said emulsion.

In one embodiment, the method for preparing an emulsion, a gelled emulsion, or a gel according to the invention further comprises at least one step of adding at least one polyol, namely maltitol.

In one embodiment, the method for preparing an emulsion, a gelled emulsion, or a gel according to the invention further comprises at least one step of adding at least one polyol, namely glycerol.

In one embodiment, the method for preparing an emulsion, a gelled emulsion, or a gel according to the invention further comprises at least one step of adding a mixture of mannitol and sorbitol.

In one embodiment, the method for preparing an emulsion, a gelled emulsion, or a gel according to the invention further comprises at least one step of adding at least one antioxidant selected from the group of the vitamin C derivatives.

In one embodiment, the method for preparing an emulsion, a gelled emulsion, or a gel according to the invention further comprises at least one step of adding at least one antioxidant selected from the group of the vitamin C derivatives comprising magnesium ascorbyl phosphate, sodium ascorbyl phosphate, ascorbyl-2-glucoside, and mixtures thereof.

In one embodiment, said at least one vitamin C derivative is magnesium ascorbyl phosphate.

In one embodiment, the method for preparing an emulsion, a gelled emulsion, or a gel according to the invention further comprises at least one step of adding at least one antioxidant selected from the group of the derivatives of vitamin E and the tocopherols.

In one embodiment, the method for preparing an emulsion, a gelled emulsion, or a gel according to the invention further comprises at least one step of adding at least one antioxidant selected from the group of the carotenoids and retinoids and derivatives thereof.

In one embodiment, the method for preparing an emulsion, a gelled emulsion, or a gel according to the invention further comprises at least one step of adding at least one antioxidant selected from the group of the carotenoids and retinoids and derivatives thereof comprising retinol, retinoic acid, retinal, retinol esters, and carotene.

In one embodiment, the method for preparing an emulsion, a gelled emulsion, or a gel according to the invention further comprises at least one step of adding at least one antioxidant selected from the group of the pseudo-tripeptides. In one embodiment, the pseudo-tripeptide is glutathione.

In one embodiment, the method for preparing an emulsion, a gelled emulsion, or a gel according to the invention further comprises at least one step of adding at least one antioxidant selected from the group comprising the different forms of coenzyme Q10, ubiquinone, or ubiquinol.

In one embodiment, the method for preparing an emulsion, a gelled emulsion, or a gel according to the invention further comprises at least one step of adding at least one vitamin.

In one embodiment, the method for preparing an emulsion, a gelled emulsion, or a gel according to the invention further comprises at least one step of adding at least one vitamin selected from the group comprising retinol, thiamine, riboflavin, nicotinamide, dexpenthenol, pyridoxine, ascorbic acid, ergocalciferol, tocopherol, biotin, and folic acid alone or as a mixture.

In one embodiment, the vitamin concentration in an emulsion according to the invention is between 0.01 and 200 mg/g.

In one embodiment, the vitamin concentration in an emulsion according to the invention is between 0.01 and 100 mg/g.

In one embodiment, the vitamin concentration in an emulsion according to the invention is between 0.5 and 50 mg/g.

In one embodiment, the method for preparing an emulsion, a gelled emulsion, or a gel according to the invention further comprises at least one step of adding at least one amino acid.

In one embodiment, the method for preparing an emulsion, a gelled emulsion, or a gel according to the invention further comprises at least one step of adding at least one amino acid selected from the group consisting of the essential amino acids, semi-essential amino acids, and non-essential amino acids, alone or as a mixture.

In one embodiment, the method for preparing an emulsion, a gelled emulsion, or a gel according to the invention further comprises at least one step of adding at least one amino acid selected from the group of the essential amino acids.

In one embodiment, the method for preparing an emulsion, a gelled emulsion, or a gel according to the invention further comprises at least one step of adding at least one essential amino acid selected from the group comprising isoleucine, leucine, lysine, methionine, phenylalanine, threonine, tryptophan, and valine, alone or as a mixture.

In one embodiment, the method for preparing an emulsion, a gelled emulsion, or a gel according to the invention further comprises at least one step of adding at least one amino acid selected from the group of the semi-essential amino acids.

In one embodiment, the method for preparing an emulsion, a gelled emulsion, or a gel according to the invention further comprises at least one step of adding at least one semi-essential amino acid selected from the group comprising arginine and histidine, alone or as a mixture.

In one embodiment, the method for preparing an emulsion, a gelled emulsion, or a gel according to the invention further comprises at least one step of adding at least one amino acid selected from the group of the non-essential amino acids.

In one embodiment, the method for preparing an emulsion, a gelled emulsion, or a gel according to the invention further comprises at least one step of adding at least one non-essential amino acid selected from the group comprising alanine, asparagine, aspartic acid, cysteine, glutamine, glutamic acid, glycine, proline, serine, tyrosine, alone or as a mixture.

In one embodiment, the method for preparing an emulsion, a gelled emulsion, or a gel according to the invention further comprises at least one step of adding at least one amino acid selected from the group comprising hydroxyproline, taurine, and ornithine, alone or as a mixture.

In one embodiment, the concentration of amino acids in an emulsion according to the invention is between 0.01 and 150 mg/g.

In one embodiment, the concentration of amino acids in an emulsion according to the invention is between 0.01 and 100 mg/g.

In one embodiment, the concentration of amino acids in an emulsion according to the invention is between 0.5 and 50 mg/g.

In one embodiment, the method for preparing an emulsion, a gelled emulsion, or a gel according to the invention further comprises at least one step of adding at least one vasoconstrictor.

In one embodiment, the method for preparing an emulsion, a gelled emulsion, or a gel according to the invention further comprises at least one step of adding at least one vasoconstrictor selected from the group comprising naphazoline, epinephrine, methoxamine, methylnorepinephrine, norepinephrine, oxymethazoline, phenylephrine, pseudoephedrine, synephrine, cirazoline, and xylomethazoline.

In one embodiment, the concentration of vasoconstrictors in an emulsion according to the invention is between 0.01 and 3 mg/g.

In one embodiment, the method for preparing an emulsion, a gelled emulsion, or a gel according to the invention further comprises at least one step of adding at least one vasodilator.

In one embodiment, the method for preparing an emulsion, a gelled emulsion, or a gel according to the invention further comprises at least one step of adding at least one vasodilator selected from the group comprising adenosine, nicotinic acid, minoxidil, and diazoxide, alone or as a mixture.

In one embodiment, the concentration of vasodilators in the gel, gelled emulsion, or emulsion according to the invention is between 0.01 and 10 mg/g.

In one embodiment, the method for preparing an emulsion, a gelled emulsion, or a gel according to the invention further comprises at least one step of adding at least one antihemorrhagic or hemostatic agent.

In one embodiment, the method for preparing an emulsion, a gelled emulsion, or a gel according to the invention further comprises at least one step of adding at least one antihemorrhagic or hemostatic agent selected from the group comprising aminocaproic acid or tranexamic acid, alone or as a mixture.

In one embodiment, the concentration of antihemorrhagic or hemostatic agents in the gel, gelled emulsion, or emulsion according to the invention is between 0.01 and 5 mg/g.

In one embodiment, the method for preparing an emulsion, a gelled emulsion, or a gel according to the invention further comprises at least one step of adding at least one antioxidant and at least one local anesthetic.

In one embodiment, the method for preparing an emulsion, a gelled emulsion, or a gel according to the invention further comprises at least one step of adding at least one antioxidant selected from the group of the polyols and at least one local anesthetic selected from the group of the amino amides.

In one embodiment, the method for preparing an emulsion, a gelled emulsion, or a gel according to the invention further comprises at least one step of adding at least one antioxidant, at least one vitamin, and at least one tissue regenerator.

In one embodiment, the method for preparing an emulsion, a gelled emulsion, or a gel according to the invention further comprises at least one step of adding at least one amino acid, at least one vitamin, and at least one tissue regenerator.

In one embodiment, the method for preparing an emulsion, a gelled emulsion, or a gel according to the invention further comprises at least one step of adding at least one antioxidant, at least one amino acid, at least one vitamin, and at least one tissue regenerator.

In one embodiment, the method for preparing an emulsion, a gelled emulsion, or a gel according to the invention further comprises at least one step of adding at least one active ingredient selected from the group consisting of heat-sensitive active ingredients.

In one embodiment, the at least one heat-sensitive active ingredient is added during the step of preparing the gels, gelled emulsions, or emulsions according to the invention, namely during the step of mixing the hydrogel and the fat or fat derivative.

In one embodiment, the method for preparing an emulsion, a gelled emulsion, or a gel according to the invention further comprises at least one step of adding a so-called heat-sensitive active ingredient of natural origin.

In one embodiment, the method for preparing an emulsion, a gelled emulsion, or a gel according to the invention further comprises at least one step of adding a so-called heat-sensitive active ingredient of synthetic origin.

In one embodiment, the method for preparing an emulsion, a gelled emulsion, or a gel according to the invention further comprises at least one step of adding at least one heat-sensitive active ingredient selected from the group consisting of the peptides, hormones, proteins, growth factors, antibodies, and vitamins, alone or as a mixture.

In one embodiment, the method for preparing an emulsion, a gelled emulsion, or a gel according to the invention further comprises at least one step of adding at least one heat-sensitive active ingredient selected from the group consisting of the peptides.

In one embodiment, the added heat-sensitive active ingredient is a peptide.

In one embodiment, the method for preparing an emulsion, a gelled emulsion, or a gel according to the invention further comprises at least one step of adding at least one heat-sensitive active ingredient selected from the group consisting of the hormones.

In one embodiment, the added heat-sensitive active ingredient is a hormone.

In one embodiment, the method for preparing an emulsion, a gelled emulsion, or a gel according to the invention further comprises at least one step of adding at least one heat-sensitive active ingredient selected from the group consisting of the proteins.

In one embodiment, the added heat-sensitive active ingredient is a protein.

In one embodiment, the method for preparing an emulsion, a gelled emulsion, or a gel according to the invention further comprises at least one step of adding at least one heat-sensitive active ingredient selected from the group consisting of the growth factors.

In one embodiment, the added heat-sensitive active ingredient is a growth factor.

In one embodiment, the method for preparing an emulsion, a gelled emulsion, or a gel according to the invention further comprises at least one step of adding at least one heat-sensitive active ingredient selected from the group consisting of the antibodies.

In one embodiment, the added heat-sensitive active ingredient is an antibody.

In one embodiment, the method for preparing an emulsion, a gelled emulsion, or a gel according to the invention further comprises at least one step of adding at least one heat-sensitive active ingredient selected from the group consisting of the vitamins.

In one embodiment, the vitamins are selected from the group consisting of retinol, thiamine, riboflavin, nicotinamide, dexpanthenol, pyridoxine, ascorbic acid, ergocalciferol, tocopherol, biotin, and folic acid, alone or as a mixture.

In one embodiment, the method for preparing an emulsion, a gelled emulsion, or a gel according to the invention further comprises at least one step of adding at least one ancillary ingredient for mesotherapy selected from the group consisting of the tissue inducers.

In one embodiment, the added ancillary ingredient for mesotherapy is a tissue inducer.

In one embodiment, the method for preparing an emulsion, a gelled emulsion, or a gel according to the invention further comprises at least one step of adding at least one ancillary ingredient for mesotherapy selected from the group consisting of calcium hydroxyapatite, L-polylactic acid (PLLA), polycaprolactone, polymethyl methacrylate (PMMA), and/or polyalkylamide. In one embodiment, the method for preparing an emulsion, a gelled emulsion, or a gel according to the invention further comprises at least one step of adding at least one vehicle for the tissue inducers.

In one embodiment, the method for preparing an emulsion, a gelled emulsion, or a gel according to the invention further comprises at least one step of adding at least one vehicle for the tissue inducers selected from the group consisting of glycerin, carboxymethyl cellulose, glycerol, polyethylene glycol, or even dextran.

The targeted applications are more particularly the following pathologies or treatments:
Facial aesthetic injections: for mesotherapy, skin rejuvenation, filling of wrinkles and fine lines, skin imperfections and defects, or volumizing (cheekbones, chin, lips, or any other part of the face, head, or scalp);
enhancement of tissue trophicity; enhancement of microvascularization, type II collagen, tissue thickness;
body volume injections: breast and buttock augmentation, G-spot augmentation, vaginoplasty, labial reconstruction, penis enlargement; or any body area requiring fat volume or skin trophicity enhancement: hands and limbs, heels and supporting areas, buttocks and intimate areas;
treatment of osteoarthritis, injection into the joint in order to replace or supplement deficient synovial fluid;
periurethral injection for the treatment of urinary incontinence due to sphincter insufficiency;
post-surgical injection in order to prevent adhesions, particularly peritoneal adhesions;
injection following presbyopia surgery using laser scleral incisions;
injection into the vitreous cavity;
injection during cataract surgery;
injection into the genitals;
injection for tissue spacing.

More particularly, in cosmetic surgery, depending on its viscoelastic and remanence properties, the emulsion according to the invention may be used:
to enhance skin hydration and quality through dermal mesotherapy of the face, neck, décolleté, or hands. In this case, the product is injected using needles with diameters varying between 22 and 32G and lengths generally varying between ⅛″ and ½″. The composition may be injected either by hand or using automatic devices such as injection pens and/or guns.
for tissue rejuvenation. In this case, the product is injected using needles with diameters varying between 22 and 32G and lengths generally varying between ⅛″ and ½″.
for filling fine, medium, or deep wrinkles, and injection using small-diameter needles (27 gauge to 30G predominantly, the lengths generally being ½″);
as a volumizer with injection either by needles of larger diameter, from 25 to 27G gauge, for example, and longer (30 to 40 mm, for example); or by means of cannulas mainly with a length of between 40 and 70 mm and a diameter of 22 to 25G; in that case, its cohesive nature will ensure its retention at the injection site.

The gel, gelled emulsion, or emulsion according to the invention also finds important application in joint surgery and in dental surgery for the filling of periodontal pockets, for example.

These examples of use are in no way limiting, the gel, the gelled emulsion, or the emulsion according to the present invention being more broadly intended for:
filling volumes;
generating spaces between certain tissues;
replacing deficient physiological fluids;
reshaping any body area.

The invention also relates to the method for preparing gels, gelled emulsions, or emulsions according to the invention, characterized in that it comprises the following steps:
a) providing fat or fat derivative,
b) providing a hydrogel,
c) the hydrogel and fat or fat derivative are mixed aseptically,
d) the mixture is emulsified until an emulsion, gelled emulsion, or homogeneous gel is obtained.

In one embodiment, the hydrogel contains an active ingredient as defined in the foregoing.

In one embodiment, an active ingredient is provided which is introduced at the time of mixing the hydrogel and the fat or fat derivative.

The fat, fat derivative, hydrogel, and active ingredients are as defined in the foregoing.

In various embodiments of the invention, the gel, gelled emulsion, or emulsion comprising different hydrogels, different fatty products or products derived from fat can be injected into the patient's body at different anatomical depths, on different areas of the body, and in different concentrations or dilutions. These different compositions and injection methods are adapted according to the area to be treated and the different needs for remodeling or enhancing the trophicity of the treated areas. It is thus possible to use different gels, gelled emulsions, or emulsions at the same time or successively for the same area or for different areas of the body.

The invention makes it possible to significantly enhance the rheology of fat for lipofilling by making it more suitable for injection. The gels, gelled emulsions, or emulsions obtained are stable and have a consistency that is close or equivalent to that of mayonnaise.

Gels, gelled emulsions, or emulsions have a high level of malleability, enabling them to be injected using very small-diameter cannulas and allowing practitioners to be extremely precise and to limit adverse effects, which are quite common in the case of fat injections for lipofilling alone.

The use of an emulsion, a gelled emulsion, or a gel according to the invention makes it possible to significantly reduce the appearance of post-operative bruising compared to the injection of fat for lipofilling alone.

In addition, this malleability makes it easier for the surgeon to perform the procedure and makes it more fluid on account of the good injectability of the emulsions.

Finally, this malleability enables the surgeon to reshape different areas of the body. It is possible to modify the implantation area or the shape of the implant once it is injected. These post-injection modifications allow the desired effects to be obtained more satisfactorily.

The injection of the emulsion, gelled emulsion, or gel according to the invention makes it possible to obtain visible effects on the skin, such as a remodeling effect or an improvement in the radiance of the skin, for example.

Visible effects on the skin are still visible at least 3 months after the injection.

Visible effects on the skin are still visible at least 6 months after the injection.

Visible effects on the skin are still visible at least 12 months after the injection.

Visible effects on the skin are still visible at least 18 months after the injection.

The invention also relates to a kit for preparing an emulsion, a gelled emulsion, or a gel of fat or fat derivative and hyaluronic acid comprising a cartridge (4) and at least 2 syringes.

In one embodiment of the kit, the cartridge (4) comprises at least one hydrogel.

In one embodiment, the kit comprises at least 3 syringes.

In one embodiment, the kit comprises at least 4 syringes.

In one embodiment, the kit comprises at least 10 syringes.

Kit 1: The syringes 1 and 2+the cartridge 4 filled with hyaluronic acid.

The syringe 1 (medium-volume syringe) is empty or full of fatty tissue; the cartridge 4 contains the hyaluronic acid gel (crosslinked or not); and the syringe 2 (large-volume syringe) is empty and its volume is equivalent to the addition of the volume of the syringe 1 and cartridge 4 (V1+V4).
1. Use the syringe 1 to collect the fat for lipofilling, or the MICROFAT or NANOFAT prepared previously (medium-sized syringe).
2. Take the cartridge 4 pre-filled with hyaluronic acid and connect the syringe 1 to the cartridge 4 using the Luer-Lock connectors 1a and 4a.
3. Add the syringe 2 (large-volume syringe V1+V4) to the second tip 4-b of the cartridge 4 and operate the plungers 1b and 2b of both syringes 1 and 2 using back and forth movements to create an emulsion between the fat for lipofilling and the hyaluronic acid gel.
4. After several back and forth movements (at least 5 movements), completely aspirate the emulsion into the syringe 2 (large-volume syringe).
5. Disconnect the syringe 2 from the cartridge, the gel, gelled emulsion, or emulsion contained in this syringe 2 is ready to be injected.
6. Fit a cannula onto the syringe 2, the tip 2a, in order to perform the injections. The choice of these cannulas is left to the surgeon's discretion depending on the area to be treated.

Optional steps can be carried out in order to allow the incorporation of active ingredients into the gel, gelled emulsion, or emulsion of hyaluronic acid and fat for lipofilling. These new steps can be incorporated at two separate times.
Implementation before step 3:
a) After several back and forth movements (at least 5 movements), completely aspirate the gel, gelled emulsion, or emulsion into the syringe 2 (large-volume syringe).
b) Disconnect the syringe 1 from the tip 4a of the cartridge and connect the syringe 5, containing the active ingredients, to this same cartridge on the tip 4b.
c) Empty the contents of the syringe 5 into the cartridge, then operate the plungers 2b and 5b of the syringes 2 and 5 to mix the gel, gelled emulsion, or emulsion with the active ingredient.
d) Draw the mixture completely into the syringe 2
   or
Implementation after step 4:
a) Disconnect the syringe 2 from the cartridge.
b) Connect together the syringe 2, containing the gel, gelled emulsion, or emulsion of hyaluronic acid and fat for lipofilling, and the syringe 5, containing the active ingredient, via a double Luer-Lock connector connected to the tips 2a and 5a of each of the two syringes.
c) Perform back and forth movements (at least 5 movements) in order to mix the gel, the gelled emulsion, or the emulsion and the active ingredient by operating the plungers 2b and 5b of each of the syringes.
d) Draw up the emulsion mixed with the active ingredient completely into the syringe 2.
e) Disconnect the syringe 2, the gel, gelled emulsion, or emulsion contained in this syringe 2 is ready to be injected.

Kit 2: The syringes 1, 2, and 3+the cartridge 4

The syringe 1 (medium-volume syringe) is used to collect fat for lipofilling or transformed fat; the syringe 3 (small-volume syringe) contains hyaluronic acid gel (crosslinked or not); the cartridge 4 and the syringe 2 (large-volume syringe) are empty.
1. Use the syringe 1 to collect fat for lipofilling (medium-sized syringe).
2. Connect the syringe 1 to the empty cartridge 4 using the connectors 1a and 4a.
3. Connect the syringe 3 (small syringe), containing the HA gel, to the empty cartridge 4 using the connectors 3a and 4b.
4. Fill empty the cartridge 4 with the contents of the syringe 3 (HA gel).
5. Disconnect the syringe 3 from the tip 4b, and connect the syringe 2 (larger syringe V1+V4) to the tip 4b using the connector 2a.
6. Perform back and forth movements on the plungers 1b and 2b of the two syringes 1 and 2 in order to create an emulsion (at least 5 movements),
7. Completely fill the syringe 2 with the contents of the syringe 1 and cartridge 4.
8. Disconnect the syringe 2 from the cartridge, the gel, gelled emulsion, or emulsion contained in the syringe 2 is ready to be injected.
9. Fit a cannula onto the syringe 2, the tip 2a, in order to perform the injections. The choice of these cannulas is left to the surgeon's discretion depending on the area to be treated.

In one embodiment, a syringe 5 may be added to kit 1 or kit 2, for example in order to incorporate an active ingredient as defined above.

Optional steps can be carried out in order to allow the incorporation of active ingredients into the gel, gelled emulsion, or emulsion of hyaluronic acid and fat for lipofilling. These new steps can be incorporated at two separate times.
Implementation after step 6:
a) Completely fill the syringe 3 with the contents of the syringe 1 and cartridge 4.
b) Disconnect the syringe 3 and connect in its place, on the tip 4b of the cartridge, the syringe 5 via the tip 5a.
c) Empty the contents of the syringe 5 into the cartridge 4.
d) Operate the plungers 2b and 5b of the syringes 2 and 5 to perform back and forth movements (at least 5 movements) in order to mix the gel, the gelled emulsion, or the emulsion and the active ingredient.
e) Completely fill the syringe 2 with the contents of the syringe 5 and cartridge 4.
   or
Implementation after step 8:
a) Disconnect the syringe 2 from the cartridge.
b) Connect together the syringe 2, containing the gel, gelled emulsion, or emulsion of hyaluronic acid and fat for lipofilling, and the syringe 5, containing the active ingredient, via a double Luer Lock connector, connected to the tips 2a and 5a of each of the two syringes.
c) Perform back and forth movements (at least 5 movements) in order to mix the gel, the gelled emulsion, or the emulsion and the active ingredient by operating the plungers 2b and 5b of each of the syringes.
d) Completely draw the gel, gelled emulsion, or emulsion mixed with the active ingredient into the syringe 2.
e) Disconnect the syringe 2, the emulsion contained in this syringe 2 is ready to be injected.

Kit 3: Two 20 ml syringes (2), two 10 ml syringes (1), ten 1 ml syringes (3), all with or without Luer Lock, and the cartridge (4).

In one embodiment, the two 20 ml syringes are used to collect fat from the patient, the two 10 ml syringes contain pure fat for lipofilling or pure transformed fat (MICROFAT or NANOFAT), the 1 ml syringes are filled with the different compositions to be injected into the patient, and the cartridge (4) contains the gel used to form the emulsions.
1. Use the two 20 ml syringes to collect fat from the patient.
2. The fat contained in the two 20 ml syringes is washed, then decanted or centrifuged to separate out the different constituents and keep only the pure fat.
3. The fat phase is then introduced into one of the two 10 ml syringes.
4. Optionally, it is possible to collect 2 ml of pure fat using two 1 ml syringes.
5. The two 10 ml syringes are used to turn pure fat into fat for lipofilling or into transformed fat.
6. Both syringes are connected by their Luer Lock tips to a fat preparation device to obtain fat for lipofilling or transformed fat.
7. Once the fat for lipofilling or transformed fat is obtained, a portion thereof is introduced into four 1 ml syringes.
8. The 10 ml syringe containing the remaining fat for lipofilling or transformed fat is then connected to the cartridge (4) containing the gel via the Luer Lock tip and the tip (4a), and a second 10 ml syringe is connected to the cartridge on the second (4b).
9. The contents of the cartridge are drawn into the syringe containing the fat for lipofilling or transformed fat, and the emulsion is created by performing back and forth movements between the two 10 ml syringes by operating their plungers (at least 5 movements).
10. After the emulsion is formed, it is drawn completely into the 10 ml syringe.
11. Four 1 ml syringes are then connected successively to the 10 ml syringe and filled with the emulsion in order to provide four syringes containing the gel, gelled emulsion, or emulsion to be injected.
12. After these different steps, the practitioner has two syringes containing pure fat, 4 syringes containing fat for lipofilling or transformed fat, and 4 syringes containing the gel, the gelled emulsion, or the emulsion according to the invention.
13. Fit a cannula onto each of the syringes shown in point 12, using a Luer Lock tip, in order to carry out injections of the different compositions defined previously. The choice of these cannulas is left to the surgeon's discretion depending on the area to be treated.

### Presentation of the Cartridge and the Different Emulsification Devices

In one embodiment, the cartridge is empty, and it allows for the mixing of the fat for lipofilling and the gel, consisting of at least one polysaccharide alone or in a mixture, free or crosslinked, in order to form the gel, the gelled emulsion, or the emulsion according to the invention.

In one embodiment, the cartridge contains only the gel consisting of at least one polysaccharide alone and/or in a mixture, free and/or crosslinked, and it allows for mixing of the fat for lipofilling and the gel in order to form the gel, the gelled emulsion, or the emulsion according to the invention.

In one embodiment, the cartridge allows static mixing of the fat for lipofilling and the gel, consisting of at least one polysaccharide alone or in a mixture, free or crosslinked, in order to form the gel, the gelled emulsion, or the emulsion according to the invention.

In one embodiment, the cartridge for static mixing of the fat for lipofilling is made up of two tips (6), mixing cartridge (I) the gel by the presence of two parallel planes (7c) and (8c), one having a single central hole (7d) and the second having a plurality of holes on the periphery (8d).

In one embodiment, the cartridge for static mixing of the fat for lipofilling and the gel is made up of two tips (9) and (10), also forming the body of the mixing cartridge (II), in which a mixing cross (11) is incorporated.

In one embodiment, the mixing cartridge (III) for static mixing of the fat for lipofilling and the gel is made up of a tip (12), a connector (14) enclosing a mixing cross (15), and a body (13) comprising a vertical plane (13c) having a plurality of holes (13d).

In one embodiment, the cartridge for static mixing of the fat for lipofilling and the gel has a volume of between 0.5 and 10 ml.

In one embodiment, the cartridge for static mixing of the fat for lipofilling and the gel has a volume of 0.5 ml.

In one embodiment, the cartridge for static mixing of the fat for lipofilling and the gel has a volume of 1 ml.

In one embodiment, the cartridge for static mixing of the fat for lipofilling and the gel has a volume of 2 ml.

In one embodiment, the cartridge for static mixing of the fat for lipofilling and the gel has a volume of 3 ml.

In one embodiment, the cartridge for static mixing of the fat for lipofilling and the gel has a volume of 4 ml.

In one embodiment, the cartridge for static mixing of the fat for lipofilling and the gel has a volume of 5 ml.

In one embodiment, the cartridge for static mixing of the fat for lipofilling and the gel has a volume of 6 ml.

In one embodiment, the cartridge for static mixing of the fat for lipofilling and the gel has a volume of 7 ml.

In one embodiment, the cartridge for static mixing of the fat for lipofilling and the gel has a volume of 8 ml.

In one embodiment, the cartridge for static mixing of the fat for lipofilling and the gel has a volume of 9 ml.

In one embodiment, the cartridge for static mixing of the fat for lipofilling and the gel has a volume of 10 ml.

In one embodiment, the cartridge for static mixing of the fat for lipofilling and the gel has a diameter of between 8 mm and 30 mm.

In one embodiment, the cartridge for static mixing of the fat for lipofilling and the gel has a diameter of 8 mm.

In one embodiment, the cartridge for static mixing of the fat for lipofilling and the gel has a diameter of 10 mm.

In one embodiment, the cartridge for static mixing of the fat for lipofilling and the gel has a diameter of 15 mm.

In one embodiment, the cartridge for static mixing of the fat for lipofilling and the gel has a diameter of 20 mm.

In one embodiment, the cartridge for static mixing of the fat for lipofilling and the gel has a diameter of 25 mm.

In one embodiment, the cartridge for static mixing of the fat for lipofilling and the gel has a diameter of 30 mm.

In one embodiment, the cartridge for static mixing of the fat for lipofilling and the gel has a length of between 45 mm and 100 mm.

In one embodiment, the cartridge for static mixing of the fat for lipofilling and the gel has a length of between 45 mm.

In one embodiment, the cartridge for static mixing of the fat for lipofilling and the gel has a length of between 50 mm.

In one embodiment, the cartridge for static mixing of the fat for lipofilling and the gel has a length of between 55 mm.

In one embodiment, the cartridge for static mixing of the fat for lipofilling and the gel has a length of between 60 mm.

In one embodiment, the cartridge for static mixing of the fat for lipofilling and the gel has a length of between 65 mm.

In one embodiment, the cartridge for static mixing of the fat for lipofilling and the gel has a length of between 70 mm.

In one embodiment, the cartridge for static mixing of the fat for lipofilling and the gel has a length of between 75 mm.

In one embodiment, the cartridge for static mixing of the fat for lipofilling and the gel has a length of between 80 mm.

In one embodiment, the cartridge for static mixing of the fat for lipofilling and the gel has a length of between 85 mm.

In one embodiment, the cartridge for static mixing of the fat for lipofilling and the gel has a length of between 90 mm.

In one embodiment, the cartridge for static mixing of the fat for lipofilling and the gel has a length of between 95 mm.

In one embodiment, the cartridge for static mixing of the fat for lipofilling and the gel has a length of between 100 mm.

### DESCRIPTION OF THE FIGURES

FIG. 1 shows a cartridge (4) and four syringes (1), (2), (3), and (5).

FIG. 2 shows an overall view of an assembled mixing cartridge (I), this mixing cartridge (I) is made up of two identical tips (6), a single-hole body (7), and a perforated body (8). The different parts making up the mixing cartridge (I) are fitted into each other.

FIG. 3 shows a sectional view of an assembled mixing cartridge (I), this view shows that the parts (7) and (8) are fitted into each other.

FIG. 4 shows a side view of a tip (6). These tips have a male Luer lock connector (6a) which enables the mixing cartridge (I) to be connected and locked onto a syringe.

FIG. 5 shows a sectional view of a tip (6). These tips (6) fit either into the perforated body (8) via a circular notch (6b) allowing the reduction in diameter (8b) of the perforated body (8) to be accommodated for, or into the perforated body (7) via a circular notch (6b) allowing the reduction in diameter (7b) of the perforated body (7) to be accommodated for.

FIG. 6 shows a front view of a tip (6).

FIG. 7 shows a side view of the single-hole body (7), which can be connected on one side to a tip (6) by means of a reduction in the external diameter (7a).

FIG. 8 shows a sectional view of the single-hole body (7), which can be connected on one side to the perforated body (8) by means of a circular notch (7b). This part has a plane perpendicular to the walls (7c) which is perforated with a hole parallel to the walls (7d). This hole is disposed on the side of the connection with a tip (6).

FIG. 9 shows a front view of the single-hole body (7), the hole (7d) being located in the center of the plane perpendicular to the walls (7c).

FIG. 10 shows a side view of the perforated body (8), which can be connected on one side to a tip (6) by means of a reduction in the external diameter (8a) and on the other side to the body a hole (7) by means of a reduction in the external diameter (8b).

FIG. 11 shows a sectional view of the perforated body (8), this part has a plane perpendicular to the walls (8c) which is perforated with a plurality of holes parallel to the walls (8d). These holes are disposed on the side of the reduction in the external diameter (8a) allowing connection with a tip (6).

FIG. 12 shows a front view of the perforated body (8), the holes (8d) being located on the periphery of the plane perpendicular to the walls (8c). This allows for alternation, and no preferential path, for the movement of fluids within the mixing cartridge (I), thus creating a static mixture of the constituents of the gels according to the invention.

FIG. 13 shows an overall view of an assembled mixing cartridge (II), this mixing cartridge (II) is made up of two separate bodies, the body (10) and the body (9). The different parts making up the mixing cartridge (II) are fitted into each other.

FIG. 14 shows a sectional view of an assembled mixing cartridge (II), this view shows that the two parts (9) and (10) forming the body of the cartridge are fitted into each other and that the device contains a mixing cross (11).

FIG. 15 shows a side view of the body (10). This body (10) has a male Luer lock connector (10a) which enables the mixing cartridge (II) to be connected and locked onto a syringe. The body (10) fits into the body (9) via a reduction in the external diameter (10b).

FIG. 16 shows a sectional view of the body (10).

FIG. 17 shows a front view of the body (10).

FIG. 18 shows a side view of the body (9). This body (9) has a male Luer lock connector (9a) which enables the mixing cartridge (II) to be connected and locked onto a syringe.

FIG. 19 shows a sectional view of the body (9). The body (9) fits into the body (10) via a circular notch (9b) to accommodate the reduction in diameter (10b) of the body (10).

FIG. 20 shows a front view of the body (9).

FIG. 21 shows a side view of the cross (11), this cross promotes the static mixing of the constituents of the gels according to the invention. This mixing cross (11) is enclosed in the mixing cartridge (II) within the space formed by the assembly of the body (10) and the body (9).

FIG. 22 shows an overall view of the cross (11).

FIG. 23 shows a front view of the cross (11).

FIG. 24 shows an overall view of an assembled mixing cartridge (III), this mixing cartridge (III) is made up of a tip (12), a perforated body (13), and a connector (14). The different parts making up the mixing cartridge (III) are fitted into each other.

FIG. 25 shows a sectional view of an assembled mixing cartridge (III), this view shows that the various parts constituting it, (12)-(13) and (14), are fitted into each other and that the device contains a mixing cross (15) at the connector (14).

FIG. 26 shows a side view of the tip (12). This tip has a male Luer lock connector (12a) which enables the mixing cartridge (III) to be connected and locked onto a syringe.

FIG. 27 shows a sectional view of the tip (12). The tip (12) fits into the perforated body (13) via a circular notch (12b) to accommodate the reduction in diameter (13b) of the perforated body (13).

FIG. 28 shows a front view of the tip (12).

FIG. 29 shows a side view of the perforated body (13), which can be connected on one side to the tip (12) by means of a reduction in the external diameter (13a) and on the other side to the connector (14) by means of a reduction in the external diameter (13b).

FIG. 30 shows a sectional view of the perforated body (13), this part has a plane perpendicular to the walls (13c) which is perforated with a plurality of holes parallel to the walls (13d). These holes are disposed on the side (13a) of the connection with the tip (12).

FIG. 31 shows a front view of the perforated body (13), the holes (13d) are located on the periphery of the plane perpendicular to the walls (13c), allowing for alternation, and no preferential path, for the movement of fluids within the mixing cartridge (III), thus creating a static mixture of the constituents of the gels according to the invention.

FIG. 32 shows a side view of the cross (15), this cross promotes the static mixing of the constituents of the gels according to the invention. This mixing cross (15) is enclosed in the mixing cartridge (III) within the space formed by the assembly of the perforated body (13) and the connector (14).

FIG. 33 shows an overall view of the cross (15).

FIG. 34 shows a front view of the cross (15).

FIG. 35 shows a side view of the connector (14). This connector (14) has a male Luer lock connector (14a) which enables the mixing cartridge (III) to be connected and locked onto a syringe.

FIG. 36 shows a sectional view of the connector (14). The connector (14) fits into the perforated body (13) via a circular notch (14b) to accommodate the reduction in diameter (13b) of the perforated body (13).

FIG. 37 shows a front view of the connector (14).

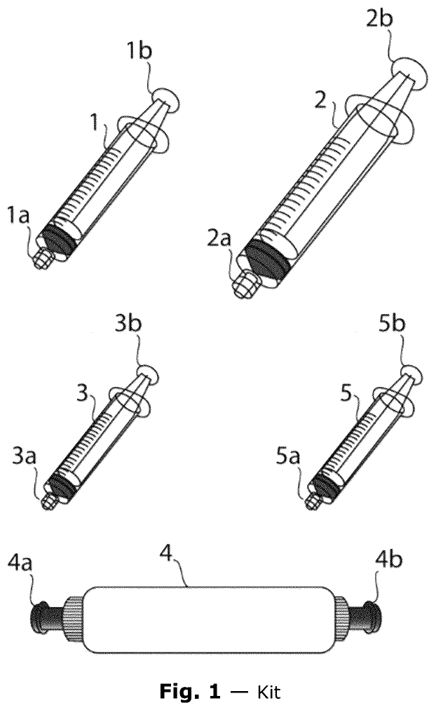

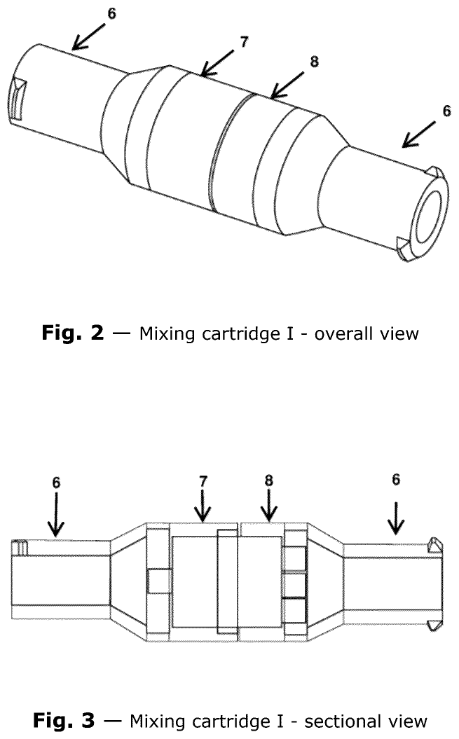

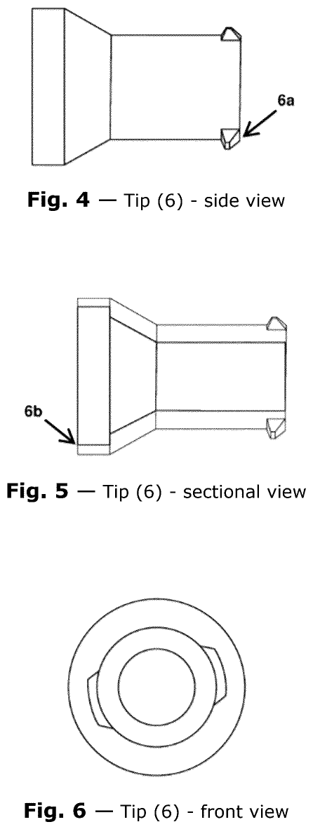

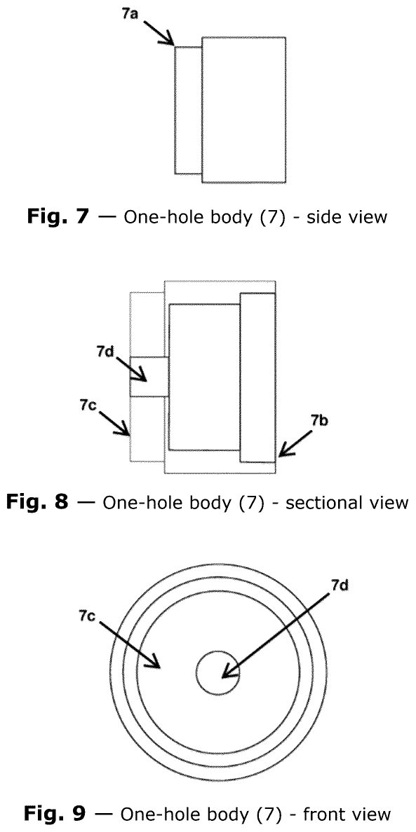

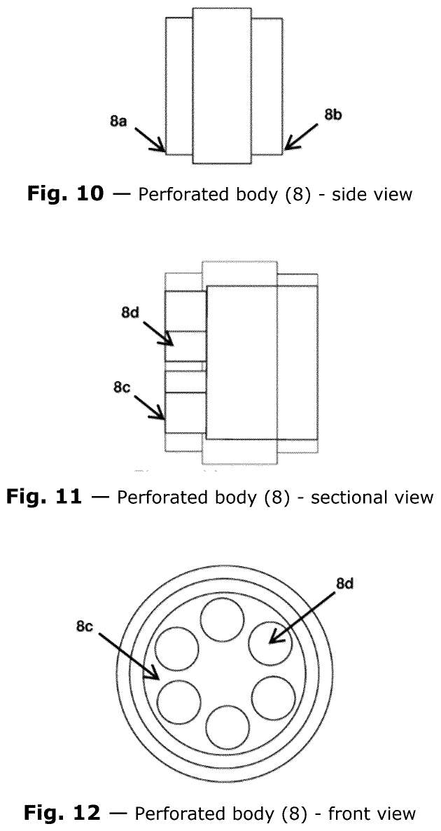

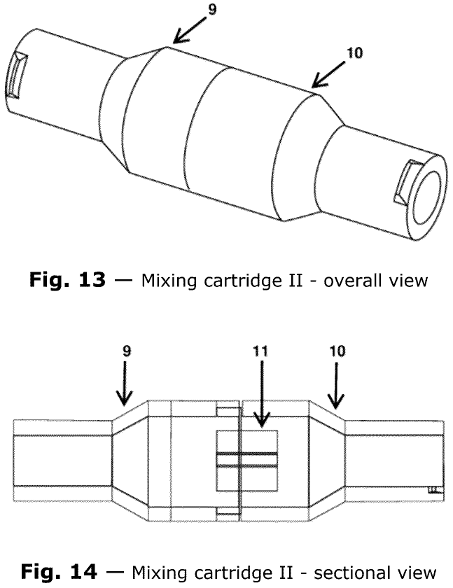

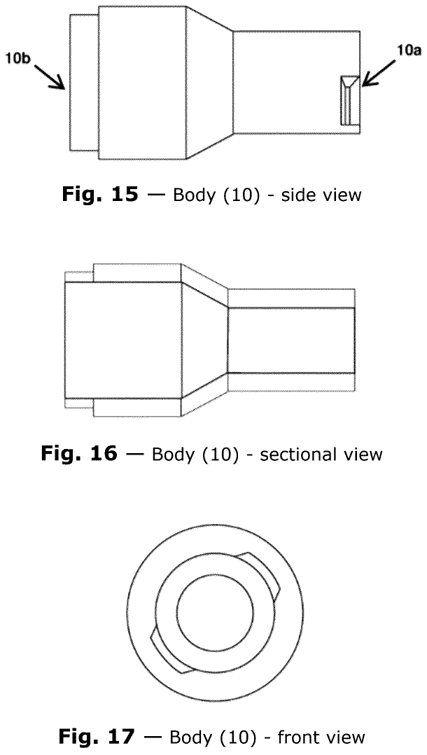

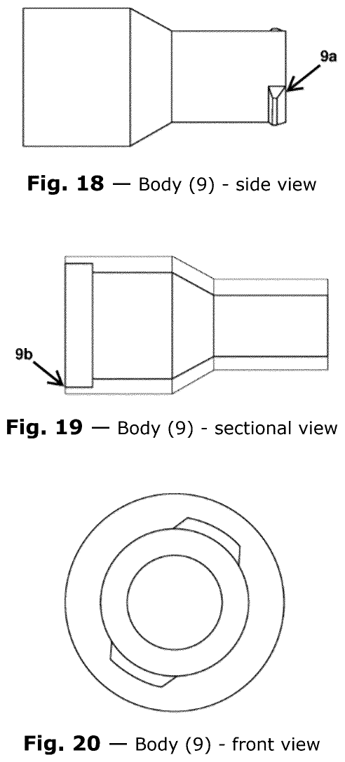

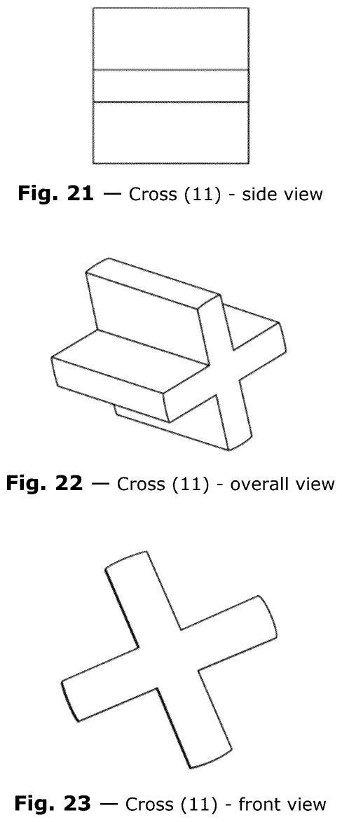

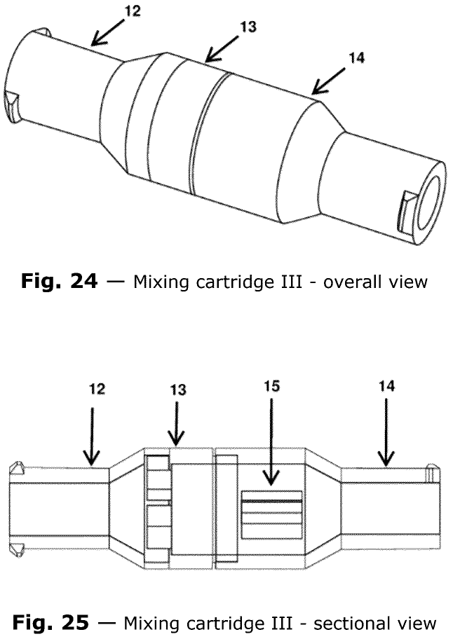

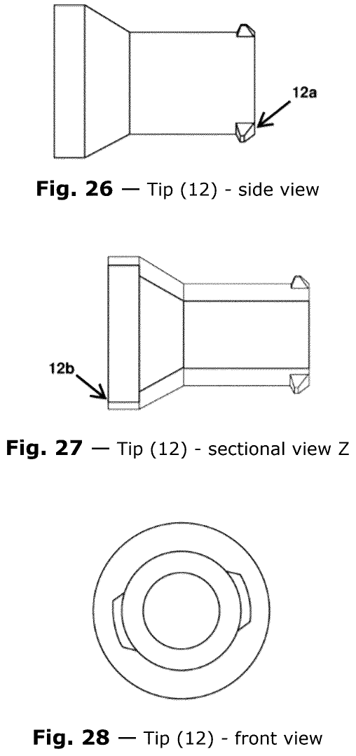

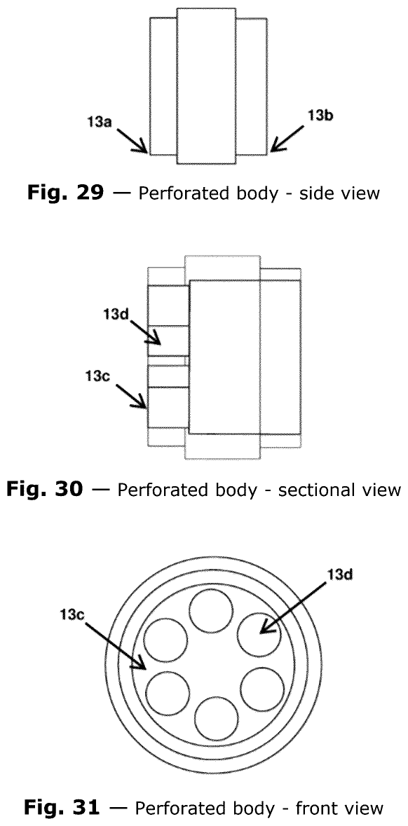

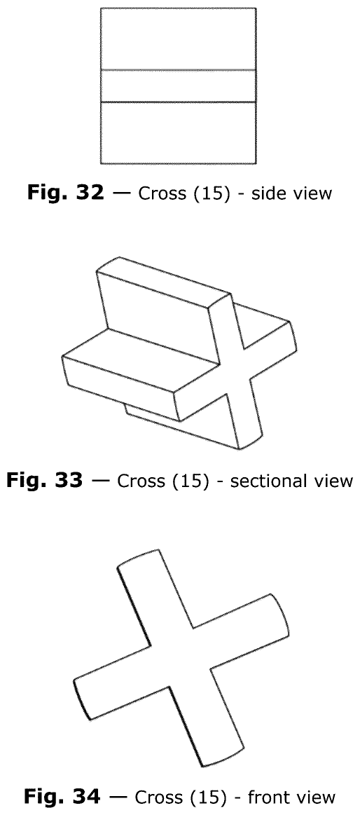

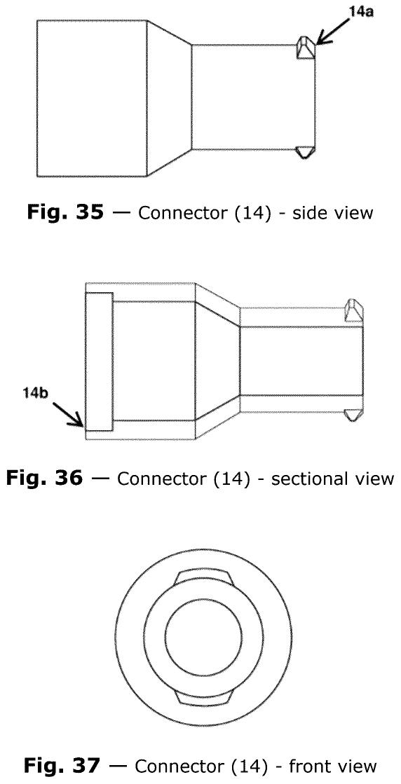

The following examples are being provided to illustrate the invention.

### EXAMPLES

### Example 1: Hyaluronic Acid Gels

A commercial non-crosslinked hyaluronic acid gel, the product Stylage® Hydro (VIVACY), and a commercial crosslinked hyaluronic acid gel comprising a local anesthetic, the product Juvederm Ultra® 2 (ALLERGAN), were used for the preparation of the fat and hyaluronic acid emulsions according to the invention. These commercial hyaluronic acid gels are available in 1 ml syringes.

### Example 2: Method for Recovering Fat Used to Prepare the Fat Emulsion

Fatty tissues, called fat for lipofilling, were harvested during surgery with appropriate donor consent, preferably from the abdomen or knees.

Fat harvesting was performed using standard surgical techniques under anesthesia according to a protocol which involves, in particular, identification of the area of the body to be sampled, making an incision in the harvesting area followed or not by an infiltration with Klein serum, harvesting of fat for lipofilling using a specific Tulip®-brand Tonnard cannula and transferring the fat from the cannula to a sterile syringe under vacuum by means of back and forth movements in the tissues and fanning movements of the cannula in order to prevent bleeding and to obtain a uniform appearance after collection.

The choice of material used for the sample as well as the technical characteristics thereof is left to the surgeon's discretion depending on the area to be treated.

The syringe filled with fat is ready to use or can be placed into cold storage before use.

### Example 3: Method for Preparing an Emulsion of Fat for Lipofilling

Recovered fat for lipofilling is collected using a sterile 5 ml syringe equipped with a Luer lock system.

This sterile syringe is connected to one of the inlets of a sterile 3-way valve or to one of the inlets of a sterile double Luer lock connector, whereas an empty sterile 5 ml syringe equipped with the Luer lock system is connected to one of the other inlets.

In order to obtain a homogeneous emulsion based solely on fat for lipofilling, the plungers are moved back and forth rapidly between the two sterile syringes connected to each other by the 3-way valve or the double Luer lock connector. At least 40 round trips are necessary in order to obtain a satisfactory emulsion of fat for lipofilling.

### Example 4: Method for Preparing an Emulsion of Fat for Lipofilling and Hyaluronic Acid According to the Invention

4 ml of recovered fat for lipofilling are collected using a sterile 5 ml syringe equipped with a Luer lock system.

This sterile syringe is connected to one of the inlets of a sterile 3-way valve or to one of the inlets of a sterile double Luer lock connector, whereas a syringe containing 1 ml of hyaluronic acid gel is connected to one of the other inlets.

The hyaluronic acid gel is then introduced into the sterile syringe containing the fat for lipofilling.

The syringe containing the hyaluronic acid gel is replaced with an empty sterile 5 ml syringe equipped with a Luer lock system.

In order to obtain a homogeneous emulsion based on fat for lipofilling and hyaluronic acid, the plungers are moved back and forth rapidly between the two sterile syringes connected to each other by the 3-way valve or the double Luer lock connector. At least 40 round trips are necessary in order to obtain a satisfactory emulsion of fat for lipofilling and hyaluronic acid.

In one variant, the homogeneous emulsion is obtained using the kit comprising the cartridge as defined above.

### Example 5: Storage Stability of an Emulsion of Fat for Lipofilling and Hyaluronic Acid According to the Invention

The stability of an emulsion of fat for lipofilling and hyaluronic acid prepared according to the method of Example 2 from the product Stylage® Hydro was monitored visually during horizontal storage on a table at 20° C., 2 h and 4 h after its preparation.

The stability of an emulsion of fat for lipofilling prepared according to the method of Example 1 was also evaluated in parallel according to the same protocol.

The results of this stability study are described in Table 1.

TABLE 1

| | | | Emulsion of fat for |
|---|---|---|---|
| | | Emulsion of fat for | lipofilling and hyaluronic |
| Composition | | lipofilling | acid |
| Observation | 2 h | Formation of two phases | Homogeneous mixture |
| | 4 h | Formation of two phases | Homogeneous mixture |

An emulsion of pure fat for lipofilling degrades rapidly, as indicated by the formation of two phases after 2 hours of storage at room temperature.

The emulsion of fat for lipofilling and hyaluronic acid according to the invention is stable at room temperature for at least 4 hours.

The stability of the emulsion of fat for lipofilling and hyaluronic acid according to the invention offers flexibility and comfort to the surgeon in its use during a surgical procedure.

### Example 6: Injectability of Emulsions of Fat for Lipofilling and Hyaluronic Acid According to the Invention

The injectability of an emulsion of fat for lipofilling and hyaluronic acid prepared according to the method of Example 2 from the product Stylage® Hydro and of an emulsion of fat for lipofilling and hyaluronic acid prepared according to the method of Example 2 from the product Juvederm Ultra® 2 was evaluated through 27G and 25G cannulas from freshly prepared emulsions and after 4 h30 of storage at 20° C.

For comparison, the injectability of a freshly prepared emulsion of fat for lipofilling was evaluated through 27G and 25G cannulas.

The results for injectability are presented in Table 2.

TABLE 2

| | | | Emulsion of fat for | | Emulsion of fat for | |
|---|---|---|---|---|---|---|
| | | | lipofilling and | | lipofilling and | |
| | | Emulsion of | hyaluronic acid from | | hyaluronic acid from | |
| | | fat for | the product Stylage ® | | the product Juvederm | |
| Composition | | lipofilling | Hydro | | Ultra ® 2 | |
| Aging of the emulsion | | Fresh | Fresh | 4 h 30 | Fresh | 4 h 30 |
| Observation | 27 G | Difficult to | Injectable | Injectable | Injectable | Injectable |
| | cannula | inject | | | | |
| | 25 G | Difficult to | Injectable | Injectable | Injectable | Injectable |
| | cannula | inject | | | | |

The results obtained show that the emulsions of fat for lipofilling and hyaluronic acid according to the invention allow the surgeon to use cannulas of smaller size than those which can be used for the injection of an emulsion of pure fat for lipofilling.

The emulsions of fat for lipofilling and hyaluronic acid according to the invention can still be injected with small size cannulas at least 4.5 hours after their preparation, which provides comfort and flexibility in terms of use during a surgical procedure.

### Example 7: Appearance of Emulsions of Fat for Lipofilling and Hyaluronic Acid According to the Invention after Passage Through a Cannula

The stability of fresh emulsions according to the invention and 4.5 hours after their preparation was evaluated by visual observation of the deposit obtained after passage through a 25G cannula.

The emulsions tested are identical to those of Example 4.

The results of the observation of these different emulsions are presented in Table 3.

TABLE 3

| | | Emulsion of fat for | | Emulsion fat for lipofilling | |
|---|---|---|---|---|---|
| | Emulsion | lipofilling and hyaluronic | | and hyaluronic acid from | |
| | of fat for | acid from the product | | the product Juvederm | |
| Composition | lipofilling | Stylage ® Hydro | | Ultra ® 2 | |
| Aging of the | Fresh | Fresh | 4 h 30 | Fresh | 4 h 30 |
| emulsion | | | | | |

| | | | |
|---|---|---|---|
| Observation | Foamy | Smooth appearance | Smooth appearance |
| | appearance | Liquid emulsion with | Emulsion without |
| | | visible spreading | apparent spreading |

The emulsions of fat for lipofilling and hyaluronic acid according to the invention have a better consistency after passing through a cannula than the emulsion of pure fat for lipofilling, even after storage for 4.5 hours at room temperature.

The emulsions of fat for lipofilling and hyaluronic acid according to the invention have a malleability that is close to that of a gel, and this characteristic is different from that of fat for lipofilling alone. The emulsions obtained according to the invention behave like a modeling paste, which is a different property from that of fat for lipofilling alone.

### Example 8: Rheological Properties of Emulsions of Fat for Lipofilling and Hyaluronic Acid According to the Invention

The viscoelastic properties were measured using a TA Instrument AR2000 rheometer. The measurements were carried out at 25° C. with a 2° 40 mm conical geometry using a frequency sweep from 0.08 to 5 Hz with a deformation of 0.8%.

The viscoelastic properties were measured for an emulsion of fat for lipofilling prepared according to Example 1 and for an emulsion of fat for lipofilling and hyaluronic acid prepared according to Example 2 from the product Stylage® Hydro.

The results of the G′ and Tan δ measurements at 1 Hz, as well as the cut-off frequency of the G′ and G″ curves, f_{cut-off}, of the different samples studied are presented in Table 4 below.

TABLE 4

| | | G′ | Tan δ | f_{cut-off} |
|---|---|---|---|---|
| Composition | Observation | (1 Hz) | (1 Hz) | (Hz) |
| Emulsion of fat for | Without prior | 6.2 Pa | 0.35 | — |
| lipofilling | injection | | | |
| Emulsion of fat for | Without prior | 22.8 Pa | 0.87 | 0.6 |
| lipofilling and | injection | | | |
| hyaluronic acid from | After injection | 22.2 Pa | 0.87 | 0.4 |
| the product | (25G cannula) | | | |
| Stylage ® Hydro | | | | |
| Stylage ® Hydro gel | Without prior | 77.7 Pa | 0.82 | 0.6 |
| | injection | | | |

The rheological properties of the emulsion of fat for lipofilling and hyaluronic acid according to the invention prepared from the Stylage® Hydro product are not affected by passage through a 25G-sized cannula.

It can be noted that the emulsion of fat for lipofilling and hyaluronic acid according to the invention prepared from the product Stylage® Hydro has a Tan δ value close to that of the commercial hyaluronic acid gel Stylage® Hydro.

The rheological properties of the emulsion of fat for lipofilling and hyaluronic acid according to the invention prepared from the Stylage® Hydro product are much more attractive than those of the emulsion of pure fat for lipofilling.

### Example 9: Microscopic Analysis of Emulsion of Fat for Lipofilling and Hyaluronic Acid According to the Invention

Microscopic analyses were carried out using a Zweiss AXIO Scope A1 optical microscope equipped with Filtrex software and with ×20 magnification.

Three types of sample were analyzed under the microscope: a sample of fat for lipofilling, a sample of an emulsion of fat for lipofilling prepared according to Example 1, and a sample of an emulsion of fat for lipofilling and hyaluronic acid according to the invention prepared according to Example 2 from the product Stylage® Hydro.

The results of the different observations of the samples under the microscope are presented in Table 5 below:

TABLE 5

| | | | Emulsion of fat for |
|---|---|---|---|
| | Fat for | Emulsion of fat | lipofilling and |
| Sample | lipofilling | for lipofilling | hyaluronic acid |
| Observations | Presence of large, | Small, unstable | Small, stable fat |
| | fatty globules | fatty globules, | globules spaced apart |
| | stuck together | stuck together | by a “protective” |
| | | with a tendency | matrix |
| | | to cluster | |
| Particle | Round | — | Round |
| shape | | | |

The emulsion of fat and hyaluronic acid according to the invention has a more uniform appearance with small fat globules incorporated in a hyaluronic acid matrix.

### Example 10: Comparative Clinical Study

Two groups A and B of patients were formed. A contained 40 patients and B contained 40, for a total of 80 patients.

Group A was injected for facial rejuvenation with an emulsion of fat for lipofilling and hyaluronic acid, while group B was injected for facial rejuvenation with fat for lipofilling.

For each patient in group A, an emulsion of fat for lipofilling taken from the patient and hyaluronic acid was prepared according to the method of Example 4, the hyaluronic acid used being the product Stylage® Hydro, with a concentration of 14 mg/g in a mannitol phosphate buffer (VIVACY Laboratory), the fat being washed and decanted fat for lipofilling of the MICROFAT type and the volume ratio of hyaluronic acid to fat being 1:4.

For each patient in group B, the fat for lipofilling is taken from the patient and is of the MICROFAT type.

On D0, each patient has fat removed from the abdominal region according to the protocol described in Example 2.

The injection into the face was performed using a TSK-brand 25G Steriglide cannula after making a pre-hole using a TSK-brand 23G Steriglide pre-hole needle.

The practitioner in charge of the injection evaluated the volume injected per hemiface and assessed the ease of injection of the product using the injectability score. This score of 0 to 3 is assessed as follows:
0: Injection impossible: the product does not come out.
1: Poor injectability: need to apply very high force to eject the product from the syringe, irregular product ejection flow and cannula clogging
2: Moderate injectability: need to apply high force to eject the product from the syringe, product ejection flow with jerking
3: Good injectability: medium to low force needed to eject the product, smooth and continuous injection, without jerking.

The person in charge of administration also assessed the malleability of the product when setting up the product using a 2-level scale:
0: The product is not malleable: it cannot be deformed and remains in a block or disperses when massaged or under digital pressure in an uncontrollable manner.
1: The product is malleable: it stays in place and by massage or digital pressure.

At D+1, the practitioner in charge of the injection assessed the adverse effects in terms of occurrence and severity, while the patient assessed the rejuvenation effect using a patient satisfaction score of 1 to 3:
1: Unsatisfactory rejuvenation effect
2: Moderately satisfactory rejuvenation effect
3: Satisfactory rejuvenation effect
4: Very satisfactory rejuvenation effect, intends to repeat

The results are summarized in Table 6 below:

TABLE 6

| Group | | | A | B | Comments |
|---|---|---|---|---|---|
| Average volume injected per hemiface | | | 2 ml | 2 ml | / |
| (ml) | | | | | |
| Injectability | 0: Injection impossible | | 0 | 0 | / |
| score | 1: Poor injectability | | 0 | 40 | The cannula |
| | | | | | gets clogged |
| | 2: Moderate injectability | | 0 | 0 | / |
| | 3: Good injectability | | 40 | 0 | / |
| Malleability | 0: The product is not | | 0 | 40 | / |
| score | malleable | | | | |
| | 1: The product is | | 40 | 0 | / |
| | malleable | | | | |
| Adverse | Occurrence | None | 0 | 0 | / |
| effects | | ≤5% | Yes: 5% | No | / |
| | | >5% | No | Yes: 50% | / |
| | Severity | None | 0 | 0 | / |
| | | Mild or | 2 | 5 | / |
| | | moderate | | | |
| | | Severe | 0 | 0 | / |

| | Type | Bruising | Bruising | / |
|---|---|---|---|---|
| | Characterization | Located at | Located at | / |
| | | the injection | the injection | |
| | | site | site | |
| Patient | 1: Unsatisfactory | 0 | 0 | / |
| satisfaction | rejuvenation effect | | | |
| score | 2: Moderately | 0 | 40 | Only a |
| | satisfactory | | | volumizing |
| | rejuvenation effect | | | effect |
| | 3: Satisfactory | 0 | 0 | / |
| | rejuvenation effect | | | |
| | 4: Very satisfactory | 40 | 0 | Enhanced |
| | rejuvenation effect | | | volume, |

reduced fine
lines, firmer
skin

These examples show that the gels according to the invention, composed of fat for lipofilling and hyaluronic acid, make it possible to obtain much more satisfactory rejuvenation results than with injections of fat for lipofilling alone.

The gels according to the invention make it possible to enhance the malleability of fat for lipofilling.

The gels according to the invention make it easier to inject fat for lipofilling because there is no problem of obstruction of the cannulas, unlike tests with fat for lipofilling alone.

Furthermore, injections of gels according to the invention make it possible to significantly limit the appearance of post-injection side effects. In fact, it appears that side effects, such as bruising, are observed in only 5% of cases with the gels according to the invention, compared to 50% in the case of injections of fat for lipofilling alone.

### Example 11: In-Vitro Injectability Study on 10 Samples

Mixture of 4 cc of fat for lipofilling, of MICROFAT or NANOFAT type, with 1 cc of Stylage® Hydro product, which is a hyaluronic acid gel with a concentration of 14 mg/g in a mannitol phosphate buffer sold by VIVACY Laboratories, the volume ratio of hyaluronic acid to fat being 1:4.

Manual injectability testing with TSK Steriglide brand cannulas and BD-brand 10 cc Luer-Lock syringes.

The choice of cannulas is made among the cannulas dedicated to the injection of filler products (22G to 27G), that is, for sizes smaller than the lipofilling cannulas (19-21G) usually used during surgical procedures.

The practitioner assessed injectability blindly and gave an injectability score using the following injectability scale:
0: Injection impossible: the product does not come out.
1: Poor injectability: need to apply very high force to eject the product from the syringe, irregular product ejection flow and cannula clogging
2: Moderate injectability: need to apply high force to eject the product from the syringe, product ejection flow with jerking
3: Good injectability: medium to low force needed to eject the product, smooth and continuous injection, without jerking

The results are summarized in Table 7 below:

TABLE 7

| Injectability Score | 27G cannula | 25G cannula | 22G cannula |
|---|---|---|---|
| MICROFAT | 0 | 0 | 1 |
| MICROFAT and | 2 | 3 | 3 |
| HYDRO emulsion | | | |
| NANOFAT | 1 | 2 | 3 |
| NANOFAT and | 3 | 3 | 3 |
| HYDRO emulsion | | | |

For an identical cannula size, the injectability is at least identical or—even more often—better with the object of the invention. This makes it possible on the one hand to perform injections with smaller cannulas, which is therefore less traumatic for the patient, and on the other hand to facilitate the practitioner's work, since the movements are more fluid and more precise.

### Example 12: Non-Comparative Clinical Study of 45 Cases

The gel studied in this example is made up of NANOFAT, resulting from the transformation of fat for lipofilling, and the product Stylage® Hydro, which is a hyaluronic acid gel with a concentration of 14 mg/g in a mannitol phosphate buffer sold by VIVACY Laboratories, the volume ratio of hyaluronic acid to fat being 1:4.

A non-comparative study of 45 clinical cases was carried out over a period of 2 years, combining a mixture of 4 cc of NANOFAT and 1 cc of Stylage® Hydro product.

On D0, each patient had fat taken from the abdominal region, then injected into the face using a 25G cannula in the malar area for facial rejuvenation. Each patient was injected on each side of the face and therefore underwent 2 injection procedures.

90 injection procedures are therefore carried out.

The results are summarized in table 8 below:

TABLE 8

| Description | Statistics |
|---|---|
| Number of patients included | 45 |
| Average age | 42 years |
| Injection area and depth | Malar, subdermal |
| Average quantity injected | 2.4 cc per side |
| Number of very satisfied | 41 |
| patients | |
| Number of satisfied patients | 4 |
| Number of dissatisfied patients | 0 |
| Patient wishing to repeat the | 43 |
| procedure | |
| Side effects | 2 patients with bruising of slight |
| | severity and limited to the injection area |
| Effect observed by patients | Skin rejuvenation, improvement of |
| | spots, reduction of fine lines, restoration |
| | of volumes, reduction of sagging skin |
| Duration of the effect | 45 patients with visible effect at 1 year |
| | No patient dissatisfaction expressed at 1 |
| | year |

This clinical study demonstrated that this novel method of skin rejuvenation gives satisfaction to all patients treated, a great majority of whom declare themselves very satisfied. Moreover, the effect is still visible more than a year after the procedure was performed. Finally, side effects are rare, limited in extent, and mild.

1.-17. (canceled)

18. An emulsion, gelled emulsion, or gel comprising a hydrogel and fat, wherein the fat is fat for lipofilling,
in that the gel, gelled emulsion, or emulsion is free of emulsifiers, free of chemical compounds promoting vascularization and/or free of polymers promoting cell viability or proliferation, such as silk fibers or collagen.

19. An emulsion, gelled emulsion, or gel comprising a hydrogel and fat, wherein the fat is fat for lipofilling,
in that the gel, gelled emulsion, or emulsion is free of emulsifiers, free of chemical compounds promoting vascularization and/or free of polymers promoting cell viability or proliferation, such as silk fibers or collagen
in that the gel, gelled emulsion, or emulsion,
and in that the volume ratio of hydrogel to fat or fat derivative is between 0.5/4.5 and 2/3.

20. An emulsion, gelled emulsion, or gel, wherein it consists of a hydrogel and fat for lipofilling,
and in that it optionally further comprises an active ingredient selected from the group consisting of local anesthetics, vitamin C derivatives, anti-inflammatoires, antioxidants, and mixtures thereof.

21. The emulsion, gelled emulsion, or gel according to claim 18, wherein fat for lipofilling is MICROFAT.

22. The emulsion, gelled emulsion, or gel according to claim 18, wherein the fat for lipofilling is NANOFAT.

23. The emulsion, gelled emulsion, or gel according to claim 18, wherein it further comprises an active ingredient selected from the group consisting of local anesthetics, vitamin C derivatives, anti-inflammatoires, antioxidants, and mixtures thereof.

24. The emulsion, gelled emulsion, or gel according to claim 18, wherein the hydrogel consists of at least one polysaccharide selected from the group comprising hyaluronic acid, keratan, heparin, cellulose, cellulose derivatives, alginic acid, xanthan, carrageenan, chitosan, chondroitin, heparosan, and biologically acceptable salts thereof, alone or as a mixture.

25. The emulsion, gelled emulsion, or gel according to claim 22, wherein said polysaccharide comprises at least one hyaluronic acid or a salt thereof, alone or as a mixture.

26. The emulsion, gelled emulsion, or gel according to claim 23, wherein said polysaccharide is hyaluronic acid in the form of sodium or potassium salt.

27. The emulsion, gelled emulsion, or gel according to claim 23, wherein said polysaccharide is non-crosslinked hyaluronic acid or a salt thereof, alone or as a mixture.

28. The emulsion, gelled emulsion, or gel according to claim 23, wherein said polysaccharide is crosslinked hyaluronic acid or a salt thereof, alone or as a mixture.

29. The emulsion, gelled emulsion, or gel according to claim 27, wherein said polysaccharide is crosslinked hyaluronic acid or a salt thereof, alone or as a mixture, said crosslinking being carried out by means of at least one bifunctional crosslinking agent, either butanediol diglycidyl ether (BDDE) or 1,2,7,8-diepoxyoctane.

30. The emulsion, gelled emulsion, or gel according to claim 23, wherein the molecular weight mw of the at least one hyaluronic acid is within a range from 0.01 MDa to 5 MDa.

31. The emulsion, gelled emulsion, or gel according to claim 23, wherein the concentration of hyaluronic acid [HA] in the hydrogel is between 2 mg/g and 50 mg/g of the total weight of said hydrogel.

32. The emulsion, gelled emulsion, or gel according to claim 18, wherein the hydrogel is sterilized, meaning that it undergoes a sterilization step after its preparation, said sterilization step being carried out by heat, by moist heat, by gamma radiation (γ), by accelerated electron beam, or by sterilizing filtration (0.22 μm) in the case of hydrogels based on non-crosslinked hyaluronic acid.

33. A method for preparing an emulsion, a gelled emulsion, or a gel according to claim 18, wherein it comprises the following steps:
a) providing fat or fat derivative,
b) providing a hydrogel,
c) the hydrogel and fat or fat derivative are mixed aseptically,
d) the mixture is emulsified until an emulsion, gelled emulsion, or homogeneous gel is obtained.

34. A kit for preparing an emulsion, a gelled emulsion, or a gel according to claim 18, wherein it comprises a cartridge and at least 2 syringes.

35. The kit according to claim 32, wherein the cartridge comprises at least one hydrogel.
